SEQUENCE LISTING (11 sequences)

SEQ ID NO: 1 - DNA, 20 bp, Artificial Sequence
  Synthetic
  misc_feature (20)..(20): (Sp18)2-chol
tccatgacgttcctgacgtt

SEQ ID NO: 2 - DNA, 20 bp, Artificial Sequence
  Synthetic
tccatgacgttcctgacgtt

SEQ ID NO: 3 - DNA, 20 bp, Artificial Sequence
  Synthetic
  misc_feature (20)..(20): (Sp18)-Cy3-(Sp18)-chol
tccatgacgttcctgacgtt

SEQ ID NO: 4 - DNA, 20 bp, Artificial Sequence
  Synthetic
  misc_feature: (Sp18)-Cy5-(Sp18)-chol
tccatgacgttcctgacgtt

SEQ ID NO: 5 - DNA, 20 bp, Artificial Sequence
  Synthetic
  misc_feature (20)..(20): Cy5
tccatgacgttcctgacgtt

SEQ ID NO: 6 - DNA, 20 bp, Artificial Sequence
  Synthetic
  misc_feature (20)..(20): (Sp18)2-chol
tccatgagcttcctgagctt

SEQ ID NO: 7 - DNA, 20 bp, Artificial Sequence
  Synthetic
tccatgagcttcctgagctt

SEQ ID NO: 8 - DNA, 24 bp, Artificial Sequence
  Synthetic
  misc_feature (24)..(24): (Sp18)2-chol
tcgtcgttttgtcgttttgtcgtt

SEQ ID NO: 9 - DNA, 24 bp, Artificial Sequence
  Synthetic
tcgtcgttttgtcgttttgtcgtt

SEQ ID NO: 10 - DNA, 24 bp, Artificial Sequence
  Synthetic
  misc_feature (24)..(24): (Sp18)2-chol
tgctgcttttgtgcttttgtgctt

SEQ ID NO: 11 - DNA, 24 bp, Artificial Sequence
  Synthetic
tgctgcttttgtgcttttgtgctt

(19) United States
(12) Patent Application Publication
(10) Pub. No.: US 2022/0370490 A1
(43) Pub. Date: Nov. 24, 2022

(54) SYNERGISTIC IMMUNOSTIMULATION THROUGH THE DUAL ACTIVATION OF TLR3/9 WITH SPHERICAL NUCLEIC ACIDS

(71) Applicant: NORTHWESTERN UNIVERSITY, Evanston, IL (US)

(72) Inventors: Chad A. Mirkin, Wilmette, IL (US); Ziyin Huang, Evanston, IL (US); Lisa Cole, Chicago, IL (US); Cassandra Elizabeth Callmann, Evanston, IL (US); Shuya Wang, Evanston, IL (US)

(21) Appl. No.: 17/749,977

(22) Filed: May 20, 2022

Related U.S. Application Data
(60) Provisional application No. 63/191,653, filed on May 21, 2021.

(51) Int. Cl. A61K 31/713; A61P 37/04; A61K 9/127

(52) U.S. Cl. CPC A61K 31/713; A61P 37/04; A61K 9/1275

(57) ABSTRACT

The present disclosure is generally directed to spherical nucleic acids (SNAs) comprising multiple TLR agonists that enable the simultaneous activation of multiple TLR pathways for maximally synergistic immune activation. In some aspects, the present disclosure provides a spherical nucleic acid (SNA) comprising: (a) a nanoparticle core; (b) one or more toll-like receptor 3 (TLR3) agonists encapsulated in the nanoparticle core; and (c) a shell of oligonucleotides attached to the external surface of the nanoparticle core, the shell of oligonucleotides comprising one or more toll-like receptor 9 (TLR9) agonist oligonucleotides. Methods of using the SNAs are also provided herein.

### CROSS-REFERENCE TO RELATED APPLICATIONS

This application claims the priority benefit under 35 U.S.C. § 119(e) of U.S. Provisional Patent Application No. 63/191,653, filed May 21, 2021, which is incorporated herein by reference in its entirety.

### STATEMENT OF GOVERNMENT INTEREST

This invention was made with government support under grant number CA199091 awarded by the National Institutes of Health (NIH). The government has certain rights in the invention.

### INCORPORATION BY REFERENCE OF MATERIAL SUBMITTED ELECTRONICALLY

This application contains, as a separate part of the disclosure, a Sequence Listing in computer-readable form which is incorporated by reference in its entirety and identified as follows: Filename: 2021-093_Seqlisting.txt; Size 2,795 bytes; Created: May 20, 2022.

### BACKGROUND

Toll-like receptors (TLRs) are a class of transmembrane signaling proteins that are integral to the innate immune system.^{1, 2} They are known for their rapid immune responses against invading microorganisms and play a significant role in promoting effective cancer immunotherapies.^{1, 3} Among the different TLR family members, TLR3, TLR7/8, and TLR9 sense nucleic acids and are generally localized and activated in the endosomes of cells.^{4, 5} TLR7/8 and TLR9 are efficiently activated by synthetic oligonucleotides with specific structural motifs that mimic the viral and/or bacterial origin of their natural agonists. Specifically, TLR7/8 is activated by guanosine (G)- and uridine (U)-rich single-stranded oligoribonucleotides,⁶ while TLR9 is potently activated by single-stranded oligodeoxynucleotides (ODNs) that contain repeating unmethylated cytosine-guanosine sequences known as a CpG motif.^{7, 8} Conversely, TLR3 binds to approximately 40-50 base-pairs (bp) of non-sequence-specific double-stranded RNA (dsRNA) for signal transduction,⁹ and it has been shown that even longer dsRNA (90 bp) may be required for TLR3 activation in the early endosome.¹⁰ Polyinosinic:polycytidylic acid [poly(I:C)] is a synthetic dsRNA that induces potent TLR3 activation and increases the cross-priming of antigen-presenting cells (APCs).¹¹⁻¹³

### SUMMARY

Toll-like receptors (TLRs) are a family of proteins that modulate the innate immune system and control the initiation of downstream immune responses. Spherical nucleic acids (SNAs) designed to stimulate single members of the TLR family (e.g., TLR7 or TLR9) have shown utility in cancer immunotherapy. The present disclosure is generally directed to SNAs synthesized with multiple TLR agonists that enable the simultaneous activation of multiple TLR pathways for maximally synergistic immune activation. The disclosure describes the synthesis and evaluation of spherical nucleic acids (SNAs) incorporating two different types of nucleic acids, synergistically activating toll-like receptors 3 and 9. These SNAs are single entity agents that enter the same target cell at defined stoichiometries, and as such allow one to control important cell signaling and regulatory processes, providing a strategy for potently activating immune cells and increasing the efficiency of their activation as highly potent immunotherapies. As shown herein, these dual-TLR activating SNAs exhibited high cellular uptake and co-delivery of the two oligonucleotides, relative to mixtures of the linear oligonucleotide counterparts. Furthermore, the dual-TLR activating SNAs augmented murine and human antigen-presenting cell maturation with prolonged duration, compared to the same amounts of oligonucleotides delivered in linear or SNA form but not conjugated to one another.

Applications of the technology disclosed herein include, but are not limited to:

Nucleic acid-based therapy

Combination therapy

Cancer immunotherapy

Advantages of the technology described herein include, but are not limited to:

Co-delivery of multiple TLR agonists nucleic acids using SNAs to the same cell

Simultaneously co-activation of multiple TLR signaling pathways for enhanced/synergistic responses

Prolong the duration of antigen-presenting cell activation, leading to efficient downstream immune responses.

In some aspects, the disclosure provides a spherical nucleic acid (SNA) comprising: (a) a nanoparticle core; (b) one or more toll-like receptor 3 (TLR3) agonists encapsulated in the nanoparticle core; and (c) a shell of oligonucleotides attached to the external surface of the nanoparticle core, the shell of oligonucleotides comprising one or more toll-like receptor 9 (TLR9) agonist oligonucleotides. In some embodiments, about 1 to about 10 TLR3 agonists are encapsulated in the nanoparticle core. In some embodiments, the TLR3 agonist is polyinosinic:polycytidylic acid (poly(I:C)), polyadenylic:polyuridylic acid (poly(A:U)), viral double-stranded RNA, or a combination thereof. In some embodiments, the shell of oligonucleotides comprises about 10 to about 100 TLR9 agonist oligonucleotides. In some embodiments, each of the one or more TLR9 agonist oligonucleotides is a CpG-motif containing oligonucleotide. In some embodiments, the SNA consists of one or more toll-like receptor 3 (TLR3) agonists encapsulated in the nanoparticle core and one or more toll-like receptor 9 (TLR9) agonist oligonucleotides in the shell of oligonucleotides. In further embodiments, an SNA of the disclosure further comprises an antigen. In some embodiments, the antigen is a peptide, a protein, or a combination thereof. In some embodiments, the antigen is a cancer-related antigen or a viral antigen. In further embodiments, the antigen is encapsulated in the nanoparticle core. In some embodiments, the antigen is a tumor cell lysate. In some embodiments, the antigen is attached to the surface of the nanoparticle core, the antigen is attached to an oligonucleotide in the shell of oligonucleotides, or both. In some embodiments, the antigen is a prostate cancer antigen. In some embodiments, the nanoparticle core is a liposomal core. In further embodiments, the liposomal core comprises a plurality of lipid groups. In some embodiments, the plurality of lipid groups comprises a lipid from the phosphatidylcholine, phosphatidylglycerol, and/or phosphatidylethanolamine families of lipids. In various embodiments, the lipid is 1,2-dioleoyl-sn-glycero-3-phosphocholine (DOPC), 1,2-dimyristoyl-sn-phosphatidylcholine (DMPC), 1-palmitoyl-2-oleoyl-sn-phosphatidylcholine (POPC), 1,2-distearoyl-sn-glycero-3-phospho-(1′-rac-glycerol) (DSPG), 1,2-dioleoyl-sn-glycero-3-phospho-(1′-rac-glycerol) (DOPG), 1,2-distearoyl-sn-glycero-3-phosphocholine (DSPC), 1,2-dipalmitoyl-sn-glycero-3-phosphocholine (DPPC), 1,2-di-(9Z-octadecenoyl)-sn-glycero-3-phosphoethanolamine (DOPE), 1,2-dihexadecanoyl-sn-glycero-3-phosphoethanolamine (DPPE), monophosphoryl Lipid A (MPLA), or a combination thereof. In some embodiments, at least one TLR9 agonist oligonucleotide in the shell of oligonucleotides is attached to the external surface of the nanoparticle core through a lipid anchor group. In some embodiments, each TLR9 agonist oligonucleotide in the shell of oligonucleotides is attached to the external surface of the nanoparticle core through a lipid anchor group. In some embodiments, the lipid anchor group is attached to the 5′ end or the 3′ end of the at least one TLR9 agonist oligonucleotide. In some embodiments, the lipid anchor group is attached to the 5′ end or the 3′ end of each TLR9 agonist oligonucleotide. In various embodiments, the lipid anchor group is tocopherol, DOPE lipid, or cholesterol. In some embodiments, the shell of oligonucleotides comprises DNA, RNA, or a combination thereof. In further embodiments, the shell of oligonucleotides comprises single-stranded DNA, double-stranded DNA, single-stranded RNA, double-stranded RNA, or a combination thereof. In some embodiments, the shell of oligonucleotides comprises one or more additional oligonucleotides. In further embodiments, the one or more additional oligonucleotides each have the same nucleotide sequence. In some embodiments, the one or more additional oligonucleotides comprises at least one oligonucleotide having a different nucleotide sequence than at least one other oligonucleotide in the one or more additional oligonucleotides. In some embodiments, a SNA of the disclosure comprises one or more additional oligonucleotides encapsulated in the nanoparticle core. In various embodiments, the one or more additional oligonucleotides encapsulated in the nanoparticle core comprises mRNA, plasmid DNA, a therapeutic oligonucleotide, a detection oligonucleotide, or a combination thereof. In some embodiments, each TLR9 agonist oligonucleotide in the shell of oligonucleotides is about 5 to about 1000 nucleotides in length. In some embodiments, each TLR9 agonist oligonucleotide in the shell of oligonucleotides is about 10 to about 50 nucleotides in length. In some embodiments, each TLR9 agonist oligonucleotide in the shell of oligonucleotides is about 20 to about 30 nucleotides in length. In some embodiments, each TLR3 agonist encapsulated in the nanoparticle core is about 5 to about 20,000 base pairs in length. In some embodiments, each TLR3 agonist encapsulated in the nanoparticle core is about 80 to about 100 base pairs in length. In further embodiments, each TLR3 agonist encapsulated in the nanoparticle core is about 100 to about 1000 base pairs in length. In some embodiments, diameter of the SNA is about 1 nanometer (nm) to about 500 nm. In further embodiments, diameter of the SNA is less than or equal to about 80 nanometers. In still further embodiments, diameter of the SNA is less than or equal to about 50 nanometers.

In some aspects, the disclosure provides a composition comprising a plurality of spherical nucleic acids (SNAs) as described herein. In some embodiments, a composition of the disclosure further comprises a therapeutic agent.

In further aspects, the disclosure provides a pharmaceutical formulation comprising a spherical nucleic acid (SNA) or composition of the disclosure, and a pharmaceutically acceptable carrier or diluent.

In some aspects, the disclosure provides an antigenic composition comprising a spherical nucleic acid (SNA), composition, or pharmaceutical formulation of the disclosure, wherein the antigenic composition is capable of generating an immune response including antibody generation or a protective immune response in a mammalian subject. In some embodiments, the antibody response is a neutralizing antibody response or a protective antibody response.

In some aspects, the disclosure provides a method of producing an immune response to cancer in a subject, comprising administering to the subject an effective amount of one or more of an antigenic composition of the disclosure, thereby producing an immune response to cancer in the subject. In some embodiments, the cancer is breast cancer, peritoneum cancer, cervical cancer, colon cancer, rectal cancer, esophageal cancer, eye cancer, liver cancer, pancreatic cancer, larynx cancer, lung cancer, skin cancer, ovarian cancer, prostate cancer, stomach cancer, testicular cancer, thyroid cancer, brain cancer, or a combination thereof.

In some aspects, the disclosure provides a method of treating a disorder comprising administering an effective amount of one or more of a spherical nucleic acid (SNA), composition, pharmaceutical formulation, or antigenic composition of the disclosure to a subject in need thereof, wherein the administering treats the disorder. In some embodiments, the disorder is cancer, an infectious disease, an autoimmune disease, or a combination thereof.

In further aspects, the disclosure provides a method of up-regulating activity of toll-like receptor 3 (TLR3) and toll-like receptor 9 (TLR9) comprising contacting a cell having TLR3 and TLR9 with a spherical nucleic acid of the disclosure. In some embodiments, the method is performed in vitro. In further embodiments, the method is performed in vivo. In still further embodiments, the method is performed ex vivo.

### BRIEF DESCRIPTION OF THE FIGURES

FIG. 1 depicts the synthesis procedure of a) poly(I:C) encapsulated liposomes and b) dual-activation SNAs as described herein.

FIG. 2 shows the characterization of dual-TLR activating SNAs. (a-b) Cryo-TEM images of (a) free poly(I:C) and (b) poly(I:C)-encapsulated liposomes. Right images show zoomed-in views of the boxed regions in the left images; (b inset): sketched view of the poly(I:C) positions. (c) Agarose gel electrophoresis of dye-labeled dual-TLR activating SNAs and their components. Red: Cy5-CpG, Green: Cy3-poly(I:C), Purple: CF-liposomes.

FIG. 3 shows the characterization procedure for determining the poly(I:C) loading in liposomes. To determine the poly(I:C) concentration, liposomes were disrupted using 0.02% Tween-20, and the absorbance at 260 nm was measured. It was observed that liposomes (empty or poly(I:C)-encapsulated) show significant absorption due to light scattering, but after disrupting with Tween-20 and subtracting the baseline (empty liposomes with Tween-20), the spectrum of the poly(I:C)-encapsulated liposomes overlaps with that of free poly(I:C). The extinction coefficient of poly(I:C) was measured to be 1,960,000 M⁻¹ cm⁻¹=14.70 L g⁻¹ cm⁻¹. The concentration of the lipid was determined using a phosphatidylcholine assay, and the concentration of the liposomes could be derived from the lipid concentration (approximately 18,737 DOPC lipids per 50 nm liposome). Then a ratio of the poly(I:C) loading per liposome could be derived from the ratio between the poly(I:C) and liposome concentrations.

FIG. 4 shows a 1% agarose gel stained with SYBR-safe nucleic acid stain showing poly(I:C) encapsulation in the liposomes. “PIC liposome” refers to poly(I:C)-encapsulated liposomes. +/− signs indicate presence/absence of 1% Triton-X in the samples. As expected, the empty liposomes and Triton-X only lanes do not show signals from the nucleic acids. For liposomes of all sizes, after incubation with Triton-X, the nucleic acid signal shifts downwards, indicating the association of poly(I:C) with the liposomes.

FIG. 5 shows (a) Hydrodynamic diameters and (b) zeta potentials of SNAs, poly(I:C)-encapsulated liposomes, and empty liposomes. Data represented as mean±SEM.

FIG. 6 shows cellular uptake of dual-TLR activating SNAs. (a) Cellular uptake of dual-TLR activating SNAs and an admixture as measured by flow cytometry. (b) Confocal microscopy images of dual-TLR activating SNAs and an admixture. Red: Cy5-CpG, Green: Cy3-poly(I:C), Purple: CF-liposomes, Blue: Nucleus. Scale bar=5 μm. (c) Comparison of the Manders overlap coefficients, calculated from confocal microscopy images, between each pair of the three components (CpG, poly(I:C), and liposomes) delivered as either SNAs or admixture. (d) Three-component overlap coefficients for the three components delivered either as SNAs or admixture. Data represented as mean±SEM. **: p<0.01, ***: p<0.001, ****: p<0.0001.

FIG. 7 shows cellular uptake of poly(I:C)-encapsulated CpG SNAs, CpG SNAs, and poly(I:C)-encapsulated liposomes after treating murine dendritic cells for 30 minutes. The Figure shows flow cytometry measurement of (a) Cy5-labeled CpG and (b) Cy3-labeled poly(I:C) signals. Data represented as mean±SEM.

FIG. 8 shows the experimental design and sample names for the immunostimulation experiments. TLR3/9 SNAs contain poly(I:C) encapsulated in liposomes with a CpG ODN shell; TLR9 SNAs are empty liposomes with a CpG ODN shell; TLR3 SNAs contain poly(I:C) encapsulated in liposomes with a GpC ODN shell. The mixture samples contain both single-agonist SNAs, a single agonist SNA and a linear TLR agonist, or both linear TLR agonists. All samples have the same CpG or poly(I:C) concentrations.

FIG. 9 shows the immune response of mouse splenocytes incubated with SNAs and TLR agonists. (a-d) Costimulatory molecule CD80 expression by (a) pDC, (b) cDC, (c) B cell, and (d) macrophage subpopulations. Data represented as mean±SEM. Statistical significance levels are with respect to TLR3/9 SNA data; *: p<0.05, **: p<0.01, ***: p<0.001, ****: p<0.0001, ns: not significant.

FIG. 10 shows the immune response of mouse splenocytes treated with SNAs and TLR agonists. Data represented as mean±SEM. Statistical significance levels are with respect to TLR3/9 SNA data; *: p<0.05, **: p<0.01, ***: p<0.001, ****: p<0.0001, ns: not significant.

FIG. 11 shows the kinetics of mouse bone marrow-derived DC activation with SNAs. (a-b) Kinetics of (a) costimulatory molecule (CD80) and (b) MHCII expression by bone marrow-derived DCs after treatment with SNAs. (c) Pearson correlation between CD80 and MHCII expression kinetics, as measured from three independent kinetics experiments. Data represented as mean±SEM. **: p<0.01.

FIG. 12 shows a comparison of the costimulatory molecule expression kinetics with that from Wang et al. (Proc. Natl. Acad. Sci. U.S.A 2019, 116, 10473-10481). The top figure shows data from DCs treated with SNAs with antigen encapsulated (SNA-E) inside the liposome, and the bottom figure shows data from DCs treated with SNAs with antigen hybridized to the oligonucleotide shell (SNA-H). It was found in Wang et al., Proc. Natl. Acad. Sci. U.S.A 2019, 116, 10473-10481 that the location of antigen changes antigen presentation kinetics but not costimulatory molecule expression kinetics, and synchronization of the two kinetics curves correlates to better tumor elimination. The costimulatory molecule expression curve from work described herein significantly overlaps with both SNA-E and SNA-H antigen presentation curves, suggesting that TLR3/9 dual-activating SNAs can be advantageous for a broader range of antigen or antigen delivery systems with different antigen presentation kinetics.

FIG. 13 shows the immune response of human PBMCs treated with SNAs and TLR agonists. (a) Costimulatory molecule CD40 expression. (b,c) Secretion levels of (b) IL-10 and (c) IFN-γ. Data represented as mean±SEM. Statistical significance levels are with respect to TLR3/9 SNA data; *: p<0.05, **: p<0.01, ***: p<0.001, ****: p<0.0001, ns: not significant; #: below the detection limit.

FIG. 14 shows the immune response of human PBMCs treated with SNAs and TLR agonists. (a-b) Costimulatory molecule (a) CD40 and (b) CD86 expression. Secretion levels of (c) TNF-α and (d) IFN-α. Data represented as mean±SEM. Statistical significance levels are with respect to TLR3/9 SNA data; *: p<0.05, **: p<0.01, ***: p<0.001, ****: p<0.0001, ns: not significant; #: below the detection limit.

FIG. 15 shows DC subpopulation activation in human PBMCs. (a-c) Costimulatory molecule CD40 expression on (a) BDCA-1 DC, (b) BDCA-2 DC, and (c) BDCA-3 DC subpopulations in human PBMCs. Data represented as mean±SEM. Statistical significance levels are with respect to TLR3/9 SNA data; *: p<0.05, **: p<0.01, ***: p<0.001, ****: p<0.0001, ns: not significant.

FIG. 16 shows costimulatory molecule CD86 expression on (a) BDCA-1 DC, (b) BDCA-2 DC, and (c) BDCA-3 DC subpopulations in human PBMCs. Data represented as mean±SEM. Statistical significance levels are with respect to TLR3/9 SNA data; *: p<0.05, **: p<0.01, ***: p<0.001, ****: p<0.0001, ns: not significant.

### DETAILED DESCRIPTION

Spherical nucleic acids (SNAs) are nanoscale constructs consisting of highly oriented oligonucleotide shells densely packed onto nanoparticle cores,^{14, 15} and have proven highly effective at TLR activation. SNAs potently stimulate immune cells when either TLR7/8¹⁶ or TLR9¹⁷⁻²⁰ targeting sequences are employed as the oligonucleotide shell. SNAs undergo scavenger receptor-mediated endocytosis, which leads to their high cellular internalization and initial endosome localization.²¹ Consequently, SNAs are attractive for targeting endosomal receptors. In addition, since SNAs are resistant to nuclease degradation and are multivalent, high-affinity TLR binders, they are more effective immunostimulating agents than conventional linear sequences.^{22, 23}

The synergistic activation of multiple TLRs may enhance the T cell responses that are required for disease protection,^{24, 25} as this triggers multiple immunostimulation pathways. However, activating TLR9 and TLR7/8 together is challenging since activation of TLR8 inhibits the activation of TLR7 and TLR9, and TLR9 activation inhibits TLR7 activation.²⁶ This may be because they share a myeloid differentiation primary response 88 (MyD88)-dependent signaling pathway.⁴ Indeed, the activation of one TLR dominates the available adaptor proteins in the cell, which is presumably why TLR7 activation is only prominent in TLR9-deficient models.²⁷ Conversely, co-activation of TLR9 with TLR3 is a more promising approach as these TLRs utilize different adaptor proteins; TLR3 signals through a TRIF-dependent and MyD88-independent pathway,⁴ so there is less interference and enhanced synergy. While both TLRs can signal the production of nuclear factor kappa-B (NF-κB) and produce inflammatory cytokines, TLR3 activation signals IRF3, which produces interferon (IFN)-β while TLR9 activation signals IRF7 which leads to the secretion of IFN-α,²⁸ known to upregulate TLR3 expression.²⁹ Thus, both help maintain the persistence of memory T cells but act through different mechanisms. Studies have shown that treatment with mixtures of CpG and poly(I:C) result in synergistic immune activation in various cell lines and animal models.^{24, 25, 30-32} While direct administration of nucleic acids may lead to side effects caused by nuclease degradation and non-specific immune activations,³³ encapsulating them into nanoparticles has shown enhanced delivery and activation.^{34, 35} With the SNA architecture described herein, treatments can lead to enhanced and sustained immunostimulation. Additionally, synchronization of antigen presentation and co-stimulation has shown to correlate with therapeutic efficacy; however, previously this could not be easily achieved. The present disclosure provides a generalized strategy to simultaneously broaden the window of expression and synchronize the kinetics of MHC and costimulatory molecule presentation, and therefore, induce more efficient immunostimulation.

Thus, in any of the aspects or embodiments of the disclosure, SNAs are provided that provide both TLR3 and TLR9 agonists in a single scaffold (TLR3/9 SNAs) that maximize their co-delivery to immune cells and thus generate synergistic immunostimulatory responses.

### Terminology

All language such as “from,” “to,” “up to,” “at least,” “greater than,” “less than,” and the like include the number recited and refer to ranges which can subsequently be broken down into sub-ranges.

A range includes each individual member. Thus, for example, a group having 1-3 members refers to groups having 1, 2, or 3 members. Similarly, a group having 6 members refers to groups having 1, 2, 3, 4, or 6 members, and so forth.

As used in this specification and the appended claims, the articles “a” and “an” refer to one or to more than one (for example, to at least one) of the grammatical object of the article.

“About” and “approximately” shall generally mean an acceptable degree of error for the quantity measured given the nature or precision of the measurements. Exemplary degrees of error are within 20-25 percent (%), for example, within 20 percent, 10 percent, 5 percent, 4 percent, 3 percent, 2 percent, or 1 percent of the stated value or range of values.

The terms “polynucleotide” and “oligonucleotide” are interchangeable as used herein.

A “subject” is a vertebrate organism. The subject can be a non-human mammal (e.g., a mouse, a rat, or a non-human primate), or the subject can be a human subject.

The terms “administering”, “administer”, “administration”, and the like, as used herein, refer to any mode of transferring, delivering, introducing, or transporting a SNA to a subject in need of treatment with such an agent. Such modes include, but are not limited to, oral, topical, intravenous, intraarterial, mucosal, intraperitoneal, intramuscular, intratumoral, parenteral, intradermal, intranasal, and subcutaneous administration. A combination of different routes of administration, separately or at the same time, is also contemplated by the disclosure.

As used herein, “treating” and “treatment” refers to any reduction in the severity and/or onset of symptoms associated with a disorder. Accordingly, “treating” and “treatment” includes therapeutic and prophylactic measures. One of ordinary skill in the art will appreciate that any degree of protection from, or amelioration of, a disorder is beneficial to a subject, such as a human patient. The quality of life of a patient is improved by reducing to any degree the severity of symptoms in a subject and/or delaying or preventing the appearance of symptoms.

An “effective amount” or a “sufficient amount” of a substance is that amount necessary to effect beneficial or desired results, including clinical results, and, as such, an “effective amount” depends upon the context in which it is being applied. For example and without limitation, an effective amount of a SNA of the disclosure is the amount that is sufficient to elicit an immune response, inhibit gene expression, and/or treat a disorder. An effective amount can be administered in one or more doses. Efficacy can be shown in an experimental or clinical trial, for example, by comparing results achieved with a substance of interest compared to an experimental control.

The term “dose” as used herein in reference to a SNA of the disclosure refers to a measured portion of the SNA (e.g., as a pharmaceutical formulation, antigenic composition, etc.) taken by (administered to or received by) a subject at any one time.

An “antigenic composition” is a composition of matter suitable for administration to a human or animal subject (e.g., in an experimental or clinical setting) that is capable of eliciting a specific immune response. In some embodiments, the immune response is elicited against an antigen, such as a cancer-related antigen or a viral antigen. As such, in some embodiments an antigenic composition includes one or more antigens or antigenic epitopes. An antigenic composition can, in some embodiments, also include one or more additional components capable of eliciting or enhancing an immune response, such as an excipient, carrier, and/or adjuvant. In certain instances, antigenic compositions are administered to elicit an immune response that protects the subject against symptoms or conditions induced by an antigen. In some cases, symptoms or disease caused by an antigen is prevented (or reduced or ameliorated) by inhibiting viral replication or expansion of cells associated with, e.g., a tumor. In the context of this disclosure, the term antigenic composition will be understood to encompass compositions that are intended for administration to a subject or population of subjects for the purpose of eliciting a protective or palliative immune response.

An “immune response” is a response of a cell of the immune system, such as a B cell, T cell, or monocyte, to a stimulus, such as an antigen (e.g., formulated as an antigenic composition or a vaccine). An immune response can be a B cell response, which results in the production of specific antibodies, such as antigen specific neutralizing antibodies. An immune response can also be a T cell response, such as a CD4+ response or a CD8+ response. B cell and T cell responses are aspects of a “cellular” immune response. An immune response can also be a “humoral” immune response, which is mediated by antibodies. In some cases, the response is specific for a particular antigen (that is, an “antigen-specific response”). A “protective immune response” is an immune response that inhibits a detrimental function or activity of an antigen, or decreases symptoms (including death) that result from the antigen. A protective immune response can be measured, for example, by immune assays using a serum sample from an immunized subject for testing the ability of serum antibodies for inhibition of tumor cell expansion, such as: ELISA-neutralization assay, antibody dependent cell-mediated cytotoxicity assay (ADCC), complement-dependent cytotoxicity (CDC), antibody dependent cell-mediated phagocytosis (ADCP), enzyme-linked immunospot (ELISpot). In addition, vaccine efficacy can be tested by measuring the T cell response CD4+ and CD8+ after immunization, using flow cytometry (FACS) analysis or ELISpot assay. The protective immune response can be tested by measuring resistance to antigen challenge in vivo in an animal model. In humans, a protective immune response can be demonstrated in a population study, comparing measurements of symptoms, morbidity, mortality, etc. in treated subjects compared to untreated controls. Exposure of a subject to an immunogenic stimulus, such as an antigen (e.g., formulated as an antigenic composition or vaccine), elicits a primary immune response specific for the stimulus, that is, the exposure “primes” the immune response. A subsequent exposure, e.g., by immunization, to the stimulus can increase or “boost” the magnitude (or duration, or both) of the specific immune response. Thus, “boosting” a preexisting immune response by administering an antigenic composition increases the magnitude of an antigen-specific response, (e.g., by increasing antibody titer and/or affinity, by increasing the frequency of antigen specific B or T cells, by inducing maturation effector function, or a combination thereof).

A “therapeutic oligonucleotide” as used herein refers to an oligonucleotide or nucleic acid that has a therapeutic effect, such as an immunostimulatory effect, a gene inhibition effect, an effect in which an immune response is inhibited, and/or a gene expression effect. Examples of therapeutic oligonucleotides include, without limitation, an inhibitory oligonucleotide, an immunostimulatory oligonucleotide (e.g., a TLR agonist), a TLR antagonist, or a nucleic acid (e.g., mRNA, plasmid DNA) that encodes a protein of interest.

As used herein, an “immunostimulatory oligonucleotide” is an oligonucleotide that can stimulate (e.g., induce or enhance) an immune response. Typical examples of immunostimulatory oligonucleotides are CpG-motif containing oligonucleotides, single-stranded RNA oligonucleotides, double-stranded RNA oligonucleotides, and double-stranded DNA oligonucleotides. A “CpG-motif” is a cytosine-guanine dinucleotide sequence. Thus, in some embodiments, a SNA of the disclosure comprises one or more TLR3 agonists encapsulated in the nanoparticle core of the SNA, one or more TLR9 agonists attached to the external surface of the nanoparticle core, and one or more additional immunostimulatory oligonucleotides encapsulated in the nanoparticle core and/or attached to the external surface of the nanoparticle core. In some embodiments, the one or more additional immunostimulatory oligonucleotides are not associated with the SNA and are administered separately, either in the same composition as the SNA or in a separate composition.

The term “inhibitory oligonucleotide” refers to an oligonucleotide that reduces the production or expression of proteins, such as by interfering with translating mRNA into proteins in a ribosome or that are sufficiently complementary to either a gene or an mRNA encoding one or more targeted proteins, that specifically bind to (hybridize with) the one or more targeted genes or mRNA thereby reducing expression or biological activity of the target protein. Inhibitory oligonucleotides include, without limitation, isolated or synthetic short hairpin RNA (shRNA or DNA), an antisense oligonucleotide (e.g., antisense RNA or DNA, chimeric antisense DNA or RNA), miRNA and miRNA mimics, small interfering RNA (siRNA), DNA or RNA inhibitors of innate immune receptors, an aptamer, a DNAzyme, or an aptazyme. In some embodiments, an inhibitory oligonucleotide is an oligonucleotide that binds to a receptor but does not activate the receptor, thereby inhibiting the receptor from further binding to a ligand and becoming activated.

All references, patents, and patent applications disclosed herein are incorporated by reference with respect to the subject matter for which each is cited, which in some cases may encompass the entirety of the document.

### Spherical Nucleic Acids (SNAs)

A “spherical nucleic acid” (SNA) as used herein comprises a spherical or substantially spherical nanoparticle core functionalized with a highly oriented oligonucleotide shell. In any of the aspects or embodiments of the disclosure, a SNA comprises (a) a nanoparticle core; (b) one or more toll-like receptor 3 (TLR3) agonists encapsulated in the nanoparticle core; and (c) a shell of oligonucleotides attached to the external surface of the nanoparticle core, the shell of oligonucleotides comprising one or more toll-like receptor 9 (TLR9) agonist oligonucleotides.

In some embodiments, the nanoparticle core is a liposomal core. Liposomal cores of the disclosure have at least a substantially spherical geometry, an internal side and an external side, and comprise a lipid bilayer. The lipid bilayer comprises, in various embodiments, a plurality of lipid groups. The plurality of lipid groups comprises, in various embodiments, a lipid from the phosphatidylcholine, phosphatidylglycerol, and/or phosphatidylethanolamine families of lipids. While not meant to be limiting, in various embodiments the lipid is 1,2-dioleoyl-sn-glycero-3-phosphocholine (DOPC), 1,2-dimyristoyl-sn-phosphatidylcholine (DMPC), 1-palmitoyl-2-oleoyl-sn-phosphatidylcholine (POPC), 1,2-distearoyl-sn-glycero-3-phospho-(1′-rac-glycerol) (DSPG), 1,2-dioleoyl-sn-glycero-3-phospho-(1′-rac-glycerol) (DOPG), 1,2-distearoyl-sn-glycero-3-phosphocholine (DSPC), 1,2-dipalmitoyl-sn-glycero-3-phosphocholine (DPPC), 1,2-di-(9Z-octadecenoyl)-sn-glycero-3-phosphoethanolamine (DOPE), 1,2-dihexadecanoyl-sn-glycero-3-phosphoethanolamine (DPPE), monophosphoryl Lipid A (MPLA), or a combination thereof.

The nanoparticle core of a SNA can range in size from about 1 nanometer (nm) to about 500 nm, about 1 nm to about 400 nm, about 1 nm to about 300 nm, about 1 nm to about 200 nm, about 1 nm to about 100 nm, about 1 nm to about 50 nm, about 10 nm to about 500 nm, about 10 nm to about 400 nm, about 10 nm to about 300 nm, about 10 nm to about 200 nm, about 10 nm to about 100 nm, about 10 nm to about 50 nm, about 10 nm to about 40 nm, about 20 nm to about 50 nm, about 20 nm to about 40 nm, about 20 nm to about 30 nm, about 10 nm to about 150 nm in diameter, about 10 nm to about 140 nm in diameter, about 10 nm to about 130 nm in diameter, about 10 nm to about 120 nm in diameter, about 10 nm to about 110 nm in diameter, about 10 nm to about 100 nm in diameter, about 10 nm to about 90 nm in diameter, about 10 nm to about 80 nm in diameter, about 10 nm to about 70 nm in diameter, about 10 nm to about 60 nm in diameter, about 10 nm to about 50 nm in diameter, about 10 nm to about 40 nm in diameter, about 10 nm to about 30 nm in diameter, or about 10 nm to about 20 nm in diameter. In other aspects, the disclosure provides a plurality of SNAs, each comprising a nanoparticle core. In these aspects, the size of the plurality of nanoparticle cores is from about 10 nm to about 150 nm (mean diameter), about 10 nm to about 140 nm in mean diameter, about 10 nm to about 130 nm in mean diameter, about 10 nm to about 120 nm in mean diameter, about 10 nm to about 110 nm in mean diameter, about 10 nm to about 100 nm in mean diameter, about 10 nm to about 90 nm in mean diameter, about 10 nm to about 80 nm in mean diameter, about 10 nm to about 70 nm in mean diameter, about 10 nm to about 60 nm in mean diameter, about 10 nm to about 50 nm in mean diameter, about 10 nm to about 40 nm in mean diameter, about 10 nm to about 30 nm in mean diameter, about 10 nm to about 20 nm in mean diameter, or about 20 nm to about 100 nm in mean diameter, about 20 nm to about 90 nm in mean diameter, about 20 nm to about 80 nm in mean diameter, about 20 nm to about 70 nm in mean diameter, about 20 nm to about 60 nm in mean diameter, about 20 nm to about 50 nm in mean diameter, about 20 nm to about 40 nm in mean diameter, or about 20 nm to about 30 nm in mean diameter. In some embodiments, the diameter (or mean diameter for a plurality of nanoparticle cores) of the nanoparticle cores is from about 10 nm to about 150 nm, from about 30 to about 100 nm, or from about 40 to about 80 nm. In some embodiments, the size of the nanoparticle cores used in a method varies as required by their particular use or application. The variation of size is advantageously used to optimize certain physical characteristics of the nanoparticles, for example, optical properties or the amount of surface area that can be functionalized as described herein. In further embodiments, a plurality of SNAs is produced and the SNAs in the plurality have a mean diameter of less than or equal to about 150 nanometers (e.g., about 10 nanometers to about 150 nanometers), or less than or equal to about 100 nanometers (e.g., about 10 nanometers to about 100 nanometers, or less than or equal to about 80 nanometers (e.g., about 10 nanometers to about 80 nanometers). In further embodiments, the nanoparticle cores in the plurality created by a method of the disclosure have a diameter or mean diameter of less than or equal to about 20 nanometers, or less than or equal to about 25 nanometers, or less than or equal to about 30 nanometers, or less than or equal to about 35 nanometers, or less than or equal to about 40 nanometers, or less than or equal to about 45 nanometers, or less than or equal to about 50 nanometers, or less than or equal to about 55 nanometers, or less than or equal to about 60 nanometers, or less than or equal to about 65 nanometers, or less than or equal to about 70 nanometers, or less than or equal to about 75 nanometers, or less than or equal to about 80 nanometers, or less than or equal to about 85 nanometers, or less than or equal to about 90 nanometers, or less than or equal to about 95 nanometers, or less than or equal to about 100 nanometers, or less than or equal to about 100 nanometers, or less than or equal to about 120 nanometers, or less than or equal to about 130 nanometers, or less than or equal to about 140 nanometers, or less than or equal to about 150 nanometers. It will be understood that any of the foregoing diameters of nanoparticle cores can apply to the diameter of the nanoparticle core itself or to the diameter of the SNA (i.e., nanoparticle core and the shell of oligonucleotides attached to the external surface of the nanoparticle core).

As described herein, in some embodiments the nanoparticle core of the SNA is a liposomal core. Liposomal particles, for example as disclosed in International Patent Application No. PCT/US2014/068429 (incorporated by reference herein in its entirety) are therefore provided by the disclosure. Liposomal particles of the disclosure have at least a substantially spherical geometry, an internal side and an external side, and comprise a plurality of lipid groups. In various embodiments, the plurality of lipid groups comprises a lipid selected from the group consisting of the phosphatidylcholine, phosphatidylglycerol, and phosphatidylethanolamine families of lipids. Lipids contemplated by the disclosure include, without limitation, 1,2-dioleoyl-sn-glycero-3-phosphocholine (DOPC), 1,2-dimyristoyl-sn-phosphatidylcholine (DMPC), 1-palmitoyl-2-oleoyl-sn-phosphatidylcholine (POPC), 1,2-distearoyl-sn-glycero-3-phospho-(1′-rac-glycerol) (DSPG), 1,2-dioleoyl-sn-glycero-3-phospho-(1′-rac-glycerol) (DOPG), 1,2-distearoyl-sn-glycero-3-phosphocholine (DSPC), 1,2-dipalmitoyl-sn-glycero-3-phosphocholine (DPPC), 1,2-di-(9Z-octadecenoyl)-sn-glycero-3-phosphoethanolamine (DOPE), 1,2-dihexadecanoyl-sn-glycero-3-phosphoethanolamine (DPPE), 1,2-dioleoyl-sn-glycero-3-phospho-(1′-rac-glycerol) (DOPG), 1,2-distearoyl-sn-glycero-3-phosphoethanolamine (DSPE), cardiolipin, lipid A, monophosphoryl Lipid A (MPLA), or a combination thereof. In various embodiments, at least one oligonucleotide in the shell of oligonucleotides is attached to the external surface of the liposomal core through a lipid anchor group. In further embodiments, the lipid anchor group is attached to the 5′ end or the 3′ end of the at least one oligonucleotide. In still further embodiments, the lipid anchor group is tocopherol or cholesterol. Thus, in various embodiments, at least one of the oligonucleotides in the shell of oligonucleotides is an oligonucleotide-lipid conjugate containing a lipid anchor group, wherein said lipid anchor group is adsorbed into the lipid bilayer. In some embodiments, all of the oligonucleotides in the shell of oligonucleotides is an oligonucleotide-lipid conjugate containing a lipid anchor group, wherein said lipid anchor group is adsorbed into the lipid bilayer. In various embodiments, 10%, 20%, 30%, 40%, 50%, 60%, 70%, 80%, 90% or 100% of the oligonucleotides in the shell of oligonucleotides is attached (e.g., adsorbed) to the external surface of the liposomal core through a lipid anchor group. The lipid anchor group comprises, in various embodiments, tocopherol, palmitoyl, dipalmitoyl, stearyl, distearyl, or cholesterol. While not meant to be limiting, any chemistry known to one of skill in the art can be used to attach the lipid anchor to the oligonucleotide, including amide linking or click chemistry. Methods of making a liposomal SNA are generally known (see, e.g., Wang, S.; Qin, L.; Yamankurt, G.; Skakuj, K.; Huang, Z.; Chen, P.-C.; Dominguez, D.; Lee, A.; Zhang, B.; Mirkin, C. A. Rational Vaccinology with Spherical Nucleic Acids. Proc. Natl. Acad. Sci. 2019, 116 (21), 10473-10481, incorporated by reference herein in its entirety).

### Oligonucleotides

The disclosure provides spherical nucleic acids (SNAs) comprising (a) a nanoparticle core; (b) one or more toll-like receptor 3 (TLR3) agonists encapsulated in the nanoparticle core; and (c) a shell of oligonucleotides attached to the external surface of the nanoparticle core, the shell of oligonucleotides comprising one or more toll-like receptor 9 (TLR9) agonist oligonucleotides. In various embodiments, the shell of oligonucleotides comprises one or more additional oligonucleotides. In some embodiments, a SNA of the disclosure comprises one or more additional oligonucleotides encapsulated in the nanoparticle core. In some embodiments, the shell of oligonucleotides comprises one or more additional oligonucleotides and one or more additional oligonucleotides is encapsulated in the nanoparticle core. In some embodiments, the one or more additional oligonucleotides each have the same nucleotide sequence. In some embodiments, the one or more additional oligonucleotides comprises at least two oligonucleotides that have a different nucleotide sequence. In some embodiments, the additional oligonucleotide is a therapeutic oligonucleotide (e.g., inhibitory oligonucleotide). In any aspects or embodiments of the disclosure, an oligonucleotide is a detection oligonucleotide that comprises a detectable marker. Oligonucleotides in the shell of oligonucleotides may be attached to a nanoparticle core through any means (e.g., covalent or non-covalent attachment). In any of the aspects or embodiments of the disclosure, the TLR3 agonist is a double-stranded nucleic acid (e.g., double-stranded RNA). Thus, each feature described herein that applies to oligonucleotides (e.g., length, modified forms) also applies to TLR3 agonists.

Oligonucleotides of the disclosure include, in various embodiments, DNA oligonucleotides, RNA oligonucleotides, modified forms thereof, or a combination thereof. In any aspects or embodiments described herein, an oligonucleotide is single-stranded, double-stranded, or partially double-stranded.

As described herein, modified forms of oligonucleotides are also contemplated by the disclosure which include those having at least one modified internucleotide linkage. In some embodiments, the oligonucleotide is all or in part a peptide nucleic acid. Other modified internucleoside linkages include at least one phosphorothioate linkage. Still other modified oligonucleotides include those comprising one or more universal bases. “Universal base” refers to molecules capable of substituting for binding to any one of A, C, G, T and U in nucleic acids by forming hydrogen bonds without significant structure destabilization. The oligonucleotide incorporated with the universal base analogues is able to function, e.g., as a probe in hybridization. Examples of universal bases include but are not limited to 5′-nitroindole-2′-deoxyriboside, 3-nitropyrrole, inosine and hypoxanthine.

The term “nucleotide” or its plural as used herein is interchangeable with modified forms as discussed herein and otherwise known in the art. The term “nucleobase” or its plural as used herein is interchangeable with modified forms as discussed herein and otherwise known in the art. Nucleotides or nucleobases comprise the naturally occurring nucleobases A, G, C, T, and U. Non-naturally occurring nucleobases include, for example and without limitations, xanthine, diaminopurine, 8-oxo-N6-methyladenine, 7-deazaxanthine, 7-deazaguanine, N4,N4-ethanocytosin, N′,N′-ethano-2,6-diaminopurine, 5-methylcytosine (mC), 5-(C3-C6)-alkynyl-cytosine, 5-fluorouracil, 5-bromouracil, pseudoisocytosine, 2-hydroxy-5-methyl-4-tr-iazolopyridin, isocytosine, isoguanine, inosine and the “non-naturally occurring” nucleobases described in Benner et al., U.S. Pat. No. 5,432,272 and Susan M. Freier and Karl-Heinz Altmann, 1997, Nucleic Acids Research, vol. 25: pp 4429-4443. The term “nucleobase” also includes not only the known purine and pyrimidine heterocycles, but also heterocyclic analogues and tautomers thereof. Further naturally and non-naturally occurring nucleobases include those disclosed in U.S. Pat. No. 3,687,808 (Merigan, et al.), in Chapter 15 by Sanghvi, in Antisense Research and Application, Ed. S. T. Crooke and B. Lebleu, CRC Press, 1993, in Englisch et al., 1991, Angewandte Chemie, International Edition, 30: 613-722 (see especially pages 622 and 623, and in the Concise Encyclopedia of Polymer Science and Engineering, J. I. Kroschwitz Ed., John Wiley & Sons, 1990, pages 858-859, Cook, Anti-Cancer Drug Design 1991, 6, 585-607, each of which are hereby incorporated by reference in their entirety). In various aspects, oligonucleotides also include one or more “nucleosidic bases” or “base units” which are a category of non-naturally-occurring nucleotides that include compounds such as heterocyclic compounds that can serve like nucleobases, including certain “universal bases” that are not nucleosidic bases in the most classical sense but serve as nucleosidic bases. Universal bases include 3-nitropyrrole, optionally substituted indoles (e.g., 5-nitroindole), and optionally substituted hypoxanthine. Other desirable universal bases include, pyrrole, diazole or triazole derivatives, including those universal bases known in the art.

Examples of oligonucleotides include those containing modified backbones or non-natural internucleoside linkages. Oligonucleotides having modified backbones include those that retain a phosphorus atom in the backbone and those that do not have a phosphorus atom in the backbone. Modified oligonucleotides that do not have a phosphorus atom in their internucleoside backbone are considered to be within the meaning of “oligonucleotide”.

Modified oligonucleotide backbones containing a phosphorus atom include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Also contemplated are oligonucleotides having inverted polarity comprising a single 3′ to 3′ linkage at the 3′-most internucleotide linkage, i.e. a single inverted nucleoside residue which may be abasic (the nucleotide is missing or has a hydroxyl group in place thereof). Salts, mixed salts and free acid forms are also contemplated. Representative United States patents that teach the preparation of the above phosphorus-containing linkages include, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, the disclosures of which are incorporated by reference herein.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts. See, for example, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, the disclosures of which are incorporated herein by reference in their entireties.

In still further embodiments, oligonucleotide mimetics wherein both one or more sugar and/or one or more internucleotide linkage of the nucleotide units are replaced with “non-naturally occurring” groups. The bases of the oligonucleotide are maintained for hybridization. In some aspects, this embodiment contemplates a peptide nucleic acid (PNA). In PNA compounds, the sugar-backbone of an oligonucleotide is replaced with an amide containing backbone. See, for example U.S. Pat. Nos. 5,539,082; 5,714,331; and 5,719,262, and Nielsen et al., Science, 1991, 254, 1497-1500, the disclosures of which are herein incorporated by reference.

In still further embodiments, oligonucleotides are provided with phosphorothioate backbones and oligonucleosides with heteroatom backbones, and including —CH₂—NH—O—CH₂—, —CH₂—N(CH₃)—O—CH₂—, —CH₂—O—N(CH₃)—CH₂—, —CH₂—N(CH₃)—N(CH₃)—CH₂— and —O—N(CH₃)—CH₂—CH₂— described in U.S. Pat. Nos. 5,489,677, and 5,602,240. Also contemplated are oligonucleotides with morpholino backbone structures described in U.S. Pat. No. 5,034,506.

In various forms, the linkage between two successive monomers in the oligonucleotide consists of 2 to 4, desirably 3, groups/atoms selected from —CH₂—, —O—, —S—, —NR^{H}—, >C═O, >C═NR^{H}, >C═S, —Si(R″)₂—, —SO—, —S(O)₂—, —P(O)₂—, —PO(BH₃)—, —P(O,S)—, —P(S)₂—, —PO(R″)—, —PO(OCH₃)—, and —PO(NHR^{H})—, where R^{H} is selected from hydrogen and C₁₋₄-alkyl, and R″ is selected from C₁₋₆-alkyl and phenyl. Illustrative examples of such linkages are —CH₂—CH₂—CH₂—, —CH₂—CO—CH₂—, —CH₂—CHOH—CH₂—, —O—CH₂—O—, —O—CH₂—CH₂—, —O—CH₂—CH═ (including R⁵ when used as a linkage to a succeeding monomer), —CH₂—CH₂—O—, —NR^{H}—CH₂—CH₂—, —CH₂—CH₂—NR^{H}—, —CH₂—NR^{H}—CH₂—, —O—CH₂—CH₂—NR^{H}—, —NR″—CO—O—, —NR^{H}—CO—NR^{H}—, —NR^{H}—CS—NR″—, —NR^{H}—C(═NR^{H})—NR^{H}—, —NR^{H}—CO—CH₂—NR^{H}—O—CO—O—, —O—CO—CH₂—O—, —O—CH₂—CO—O—, —CH₂—CO—NR^{H}—, —O—CO—NR^{H}—, —NR^{H}—CO—CH₂—, —O—CH₂—CO—NR^{H}—, —O—CH₂—CH₂—NR^{H}—, —CH═N—O—, —CH₂—NR^{H}—O—, —CH₂—O—N═ (including R⁵ when used as a linkage to a succeeding monomer), —CH₂—O—NR^{H}—, —CO—NR^{H}—CH₂—, —CH₂—NR^{H}—O—, —CH₂—NR^{H}—CO—, —O—NR^{H}—CH₂—, —O—NR^{H}, —O—CH₂—S—, —S—CH₂—O—, —CH₂—CH₂—S—, —O—CH₂—CH₂—S—, —S—CH₂—CH═ (including R⁵ when used as a linkage to a succeeding monomer), —S—CH₂—CH₂—, —S—CH₂—CH₂—O—, —S—CH₂—CH₂—S—, —CH₂—S—CH₂—, —CH₂—SO—CH₂—, —CH₂—SO₂—CH₂—, —O—SO—O—, —O—S(O)₂—O—, —O—S(O)₂—CH₂—, —O—S(O)₂—NR^{H}—, —NR^{H}—S(O)₂—CH₂—; —O—S(O)₂—CH₂—, —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —O—P(S)₂—S—, —S—P(O)₂—S—, —S—P(O,S)—S—, —S—P(S)₂—S—, —O—PO(R″)—O—, —O—PO(OCH₃)—O—, —O—PO(OCH₂CH₃)—O—, —O—PO(OCH₂CH₂S—R)—O—, —O—PO(BH₃)—O—, —O—PO(NHR^{N})—O—, —O—P(O)₂—NR^{H}H—, —NR^{H}—P(O)₂—O—, —O—P(O,NR^{H})—O—, —CH₂—P(O)₂—O—, —O—P(O)₂—CH₂—, and —O—Si(R″)₂—O—; among which —CH₂—CO—NR^{H}—, —CH₂—NR^{H}—O—, —S—CH₂—O—, —O—P(O)₂—O—O—P(—O,S)—O—, —O—P(S)₂—O—, —NR^{H} P(O)₂—O—, —O—P(O,NR^{H})—O—, —O—PO(R″)—O—, —O—PO(CH₃)—O—, and —O—PO(NHR^{N})—O—, where R^{H} is selected form hydrogen and C₁₋₄-alkyl, and R″ is selected from C₁₋₆-alkyl and phenyl, are contemplated. Further illustrative examples are given in Mesmaeker et. al., Current Opinion in Structural Biology 1995, 5, 343-355 and Susan M. Freier and Karl-Heinz Altmann, Nucleic Acids Research, 1997, vol 25, pp 4429-4443.

Still other modified forms of oligonucleotides are described in detail in U.S. patent application No. 20040219565, the disclosure of which is incorporated by reference herein in its entirety.

Modified oligonucleotides may also contain one or more substituted sugar moieties. In certain aspects, oligonucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Other embodiments include O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃]2, where n and m are from 1 to about 10. Other oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH2, heterocycloalkyl, heterocycloalkaryl, aminoalkylamino, polyalkylamino, substituted silyl, or an RNA cleaving group. In one aspect, a modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. Other modifications include 2′-dimethylaminooxyethoxy, i.e., a O(CH₂)₂ON(CH₃)₂ group, also known as 2′-DMAOE, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂.

Still other modifications include 2′-methoxy (2′-O—CH₃), 2′-aminopropoxy (2′-OCH₂CH₂CH₂NH₂), 2′-allyl (2′-CH₂—CH═CH₂), 2′-O-allyl (2′-O—CH₂—CH═CH₂) and 2′-fluoro (2′-F). The 2′-modification may be in the arabino (up) position or ribo (down) position. In one aspect, a 2′-arabino modification is 2′-F. Similar modifications may also be made at other positions on the oligonucleotide, for example, at the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. See, for example, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, the disclosures of which are incorporated by reference in their entireties herein.

In some aspects, a modification of the sugar includes Locked Nucleic Acids (LNAs) in which the 2′-hydroxyl group is linked to the 3′ or 4′ carbon atom of the sugar ring, thereby forming a bicyclic sugar moiety. The linkage is in certain aspects is a methylene (—CH₂—)ₙ group bridging the 2′ oxygen atom and the 4′ carbon atom wherein n is 1 or 2. LNAs and preparation thereof are described in WO 98/39352 and WO 99/14226.

Modified nucleotides are described in EP 1 072 679 and WO 97/12896, the disclosures of which are incorporated herein by reference. Modified nucleobases include without limitation, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified bases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzox-azin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone. Additional nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., 1991, Angewandte Chemie, International Edition, 30: 613, and those disclosed by Sanghvi, Y. S., Chapter 15, Antisense Research and Applications, pages 289-302, Crooke, S. T. and Lebleu, B., ed., CRC Press, 1993. Certain of these bases are useful for increasing binding affinity and include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. and are, in certain aspects combined with 2′-O-methoxyethyl sugar modifications. See, U.S. Pat. Nos. 3,687,808, 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,750,692 and 5,681,941, the disclosures of which are incorporated herein by reference.

Methods of making polynucleotides of a predetermined sequence are well-known. See, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are preferred for both polyribonucleotides and polydeoxyribonucleotides (the well-known methods of synthesizing DNA are also useful for synthesizing RNA). Polyribonucleotides can also be prepared enzymatically. Non-naturally occurring nucleobases can be incorporated into the polynucleotide, as well. See, e.g., U.S. Pat. No. 7,223,833; Katz, J. Am. Chem. Soc., 74:2238 (1951); Yamane, et al., J. Am. Chem. Soc., 83:2599 (1961); Kosturko, et al., Biochemistry, 13:3949 (1974); Thomas, J. Am. Chem. Soc., 76:6032 (1954); Zhang, et al., J. Am. Chem. Soc., 127:74-75 (2005); and Zimmermann, et al., J. Am. Chem. Soc., 124:13684-13685 (2002).

In various aspects, an oligonucleotide of the disclosure (e.g., a TLR9 agonist oligonucleotide), or a modified form thereof, is generally about 5 nucleotides to about 5000 nucleotides in length. More specifically, an oligonucleotide of the disclosure is about 5 to about 4000 nucleotides in length, about 5 to about 3000 nucleotides in length, about 5 to about 2000 nucleotides in length, about 5 to about 1000 nucleotides in length, about 5 to about 900 nucleotides in length, about 5 to about 800 nucleotides in length, about 5 to about 700 nucleotides in length, about 5 to about 600 nucleotides in length, about 5 to about 500 nucleotides in length about 5 to about 450 nucleotides in length, about 5 to about 400 nucleotides in length, about 5 to about 350 nucleotides in length, about 5 to about 300 nucleotides in length, about 5 to about 250 nucleotides in length, about 5 to about 200 nucleotides in length, about 5 to about 150 nucleotides in length, about 5 to about 100, about 5 to about 90 nucleotides in length, about 5 to about 80 nucleotides in length, about 5 to about 70 nucleotides in length, about 5 to about 60 nucleotides in length, about 5 to about 50 nucleotides in length about 5 to about 45 nucleotides in length, about 5 to about 40 nucleotides in length, about 5 to about 35 nucleotides in length, about 5 to about 30 nucleotides in length, about 5 to about 25 nucleotides in length, about 5 to about 20 nucleotides in length, about 5 to about 15 nucleotides in length, about 5 to about 10 nucleotides in length, about 10 to about 4000 nucleotides in length, about 10 to about 3000 nucleotides in length, about 10 to about 2000 nucleotides in length, about 10 to about 1000 nucleotides in length, about 10 to about 900 nucleotides in length, about 10 to about 800 nucleotides in length, about 10 to about 700 nucleotides in length, about 10 to about 600 nucleotides in length, about 10 to about 500 nucleotides in length about 10 to about 450 nucleotides in length, about 10 to about 400 nucleotides in length, about 10 to about 350 nucleotides in length, about 10 to about 300 nucleotides in length, about 10 to about 250 nucleotides in length, about 10 to about 200 nucleotides in length, about 10 to about 150 nucleotides in length, about 10 to about 100 nucleotides in length, about 10 to about 90 nucleotides in length, about 10 to about 80 nucleotides in length, about 10 to about 70 nucleotides in length, about 10 to about 60 nucleotides in length, about 10 to about 50 nucleotides in length about 10 to about 45 nucleotides in length, about 10 to about 40 nucleotides in length, about 10 to about 35 nucleotides in length, about 10 to about 30 nucleotides in length, about 10 to about 25 nucleotides in length, about 10 to about 20 nucleotides in length, about 10 to about 15 nucleotides in length, about 18 to about 28 nucleotides in length, about 15 to about 26 nucleotides in length, and all oligonucleotides intermediate in length of the sizes specifically disclosed to the extent that the oligonucleotide is able to achieve the desired result. In further embodiments, an oligonucleotide of the disclosure is about 5 to about 100 nucleotides in length, about 5 to about 90 nucleotides in length, about 5 to about 80 nucleotides in length, about 5 to about 70 nucleotides in length, about 5 to about 60 nucleotides in length, about 5 to about 50 nucleotides in length, about 5 to about 40 nucleotides in length, about 5 to about 30 nucleotides in length, about 5 to about 20 nucleotides in length, about 5 to about 10 nucleotides in length, and all oligonucleotides intermediate in length of the sizes specifically disclosed to the extent that the oligonucleotide is able to achieve the desired result. Accordingly, in various embodiments, an oligonucleotide of the disclosure is or is at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 300, 400, 500, 600, 700, 800, 900, 1000, 2000, 3000, 4000, or more nucleotides in length. In further embodiments, an oligonucleotide of the disclosure is less than 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, 1500, 2000, 2500, 3000, 4000, or more nucleotides in length. It will be understood that each of the foregoing lengths can refer to the length in nucleotides (for example, when referring to single-stranded nucleic acids) or the length in base pairs (for example, when referring to double-stranded nucleic acids such as TLR3 agonists). In some embodiments, each TLR9 agonist oligonucleotide in the shell of oligonucleotides is about 5 to about 1000 nucleotides in length. In some embodiments, each TLR9 agonist oligonucleotide in the shell of oligonucleotides is about 10 to about 50 nucleotides in length. In some embodiments, each TLR9 agonist oligonucleotide in the shell of oligonucleotides is about 18 to about 30 nucleotides in length. In some embodiments, each TLR3 agonist encapsulated in the nanoparticle core is about 5 to about 20,000 base pairs in length. In some embodiments, each TLR3 agonist encapsulated in the nanoparticle core is about 5 to about 5000 base pairs in length. In some embodiments, each TLR3 agonist encapsulated in the nanoparticle core is about 5 to about 20,000 base pairs in length, about 5 to about 15,000 base pairs in length, about 5 to about 10,000 base pairs in length, about 5 to about 5,000 base pairs in length, about 5 to about 1,000 base pairs in length, about 5 to about 500 base pairs in length, about 5 to about 100 base pairs in length, about 5 to about 50 base pairs in length, or about 5 to about 20 base pairs in length. In some embodiments, each TLR3 agonist encapsulated in the nanoparticle core is about 80 to about 100 base pairs in length. In further embodiments, each TLR3 agonist encapsulated in the nanoparticle core is about 100 to about 20,000 base pairs in length, about 100 to about 15,000 base pairs in length, about 100 to about 10,000 base pairs in length, about 100 to about 5000 base pairs in length, about 100 to about 2000 base pairs in length, about 100 to about 1000 base pairs in length, or about 100 to about 900 base pairs in length, or about 100 to about 800 base pairs in length, or about 100 to about 700 base pairs in length, or about 100 to about 600 base pairs in length, or about 100 to about 500 base pairs in length, or about 100 to about 450 base pairs in length, or about 100 to about 400 base pairs in length, or about 100 to about 350 base pairs in length, or about 100 to about 300 base pairs in length, or about 100 to about 250 base pairs in length, or about 100 to about 200 base pairs in length, or about 100 to about 150 base pairs in length. In some embodiments, one or more TLR3 agonists encapsulated in the nanoparticle core is at least about 45 base pairs in length. In some embodiments, one or more TLR3 agonists encapsulated in the nanoparticle core is at least about 50 base pairs in length. In some embodiments, each TLR3 agonist encapsulated in the nanoparticle core is at least 90 base pairs in length. In various embodiments, the shell of oligonucleotides attached to the external surface of the nanoparticle core of the SNA comprises a plurality of oligonucleotides that all have the same length/sequence, while in some embodiments, the plurality of oligonucleotides comprises one or more oligonucleotide that have a different length and/or sequence relative to at least one other oligonucleotide in the plurality. In some embodiments, the shell of oligonucleotides comprises a plurality of oligonucleotides comprising TLR9 agonist oligonucleotides. In some embodiments, the shell of oligonucleotides comprises a plurality of oligonucleotides comprising TLR9 agonist oligonucleotides and one or more additional oligonucleotides (e.g., therapeutic oligonucleotides). In some embodiments, the shell of oligonucleotides consists of a plurality of TLR9 agonist oligonucleotides. In various embodiments, the shell of oligonucleotides comprises or consists of one type of TLR9 agonist, while in some embodiments the shell of oligonucleotides comprises more than one type of TLR9 agonist. Types of TLR9 agonists include, without limitation, CpG oligonucleotides (e.g., class-A CpG, a mouse class-B CpG, a human class-B CpG, or a combination thereof). In further embodiments, the nanoparticle core comprises or consists of one or more TLR3 agonists encapsulated therein. In various embodiments, the nanoparticle core comprises or consists of one type of TLR3 agonist, while in some embodiments the nanoparticle core comprises more than one type of TLR3 agonist. In various embodiments, the one or more TLR3 agonists encapsulated in the nanoparticle core all have the same length/sequence, while in some embodiments, one or more of the TLR3 agonists has a different length and/or sequence relative to at least one other TLR3 agonist. Types of TLR3 agonists include, without limitation, polyinosinic:polycytidylic acid (poly(I:C)), polyadenylic:polyuridylic acid (poly(A:U)), double-stranded RNA, viral double stranded RNA, or a combination thereof. In some embodiments, the nanoparticle core comprises one or more TLR3 agonists and one or more additional oligonucleotides (e.g., therapeutic oligonucleotides, inhibitory oligonucleotides, mRNA, plasmid DNA) encapsulated therein.

Spacers. In some aspects and embodiments, one or more oligonucleotides in the shell of oligonucleotides that is attached to the nanoparticle core of a SNA comprise a spacer. “Spacer” as used herein means a moiety that serves to increase distance between the nanoparticle core and the oligonucleotide, or to increase distance between individual oligonucleotides when attached to the nanoparticle core in multiple copies, or to improve the synthesis of the SNA. Thus, spacers are contemplated being located between an oligonucleotide and the nanoparticle core. In some embodiments, an oligonucleotide encapsulated in the nanoparticle core of a SNA comprises a spacer.

In some aspects, the spacer when present is an organic moiety. In some aspects, the spacer is a polymer, including but not limited to a water-soluble polymer, a nucleic acid, a polypeptide, an oligosaccharide, a carbohydrate, a lipid, an ethylglycol, or a combination thereof. In any of the aspects or embodiments of the disclosure, the spacer is an oligo(ethylene glycol)-based spacer. In various embodiments, an oligonucleotide comprises 1, 2, 3, 4, 5, or more spacer (e.g., Spacer-18 (hexaethyleneglycol)) moieties. In further embodiments, the spacer is an alkane-based spacer (e.g., C12). In some embodiments, the spacer is an oligonucleotide spacer (e.g., T5). An oligonucleotide spacer may have any sequence that does not interfere with the ability of the oligonucleotides to become bound to the nanoparticle core or to a target. In certain aspects, the bases of the oligonucleotide spacer are all adenylic acids, all thymidylic acids, all cytidylic acids, all guanylic acids, all uridylic acids, or all some other modified base.

In various embodiments, the length of the spacer is or is equivalent to at least about 2 nucleotides, at least about 3 nucleotides, at least about 4 nucleotides, at least about 5 nucleotides, 5-10 nucleotides, 10 nucleotides, 10-30 nucleotides, or even greater than 30 nucleotides.

Density. The number of oligonucleotides on the external surface of a SNA and the number of TLR3 agonists (and optional additional oligonucleotide(s), e.g., mRNA, plasmid DNA, therapeutic oligonucleotide(s)) encapsulated in a nanoparticle core can vary. In general, a surface density adequate to make the nanoparticles stable and the conditions necessary to obtain it for a desired combination of nanoparticles and oligonucleotides can be determined empirically. The number and length of TLR3 agonists (and optional additional oligonucleotide(s)) encapsulated in a nanoparticle core may be determined to an extent by the diameter of the nanoparticle core. Similarly, the number of TLR9 agonists (and optional additional oligonucleotide(s)) on the external surface of a SNA may be determined to an extent by the diameter of the nanoparticle core and/or the nucleotide sequence of the TLR9 agonist. Generally, a surface density of at least about 2 pmoles/cm² will be adequate to provide stable nanoparticle-oligonucleotide compositions. In some aspects, the surface density is at least 15 pmoles/cm². Methods are also provided wherein the oligonucleotide is attached to the external surface of the nanoparticle core at a surface density of at least 2 pmol/cm², at least 3 pmol/cm², at least 4 pmol/cm², at least 5 pmol/cm², at least 6 pmol/cm², at least 7 pmol/cm², at least 8 pmol/cm², at least 9 pmol/cm², at least 10 pmol/cm², at least about 15 pmol/cm², at least about 19 pmol/cm², at least about 20 pmol/cm², at least about 25 pmol/cm², at least about 30 pmol/cm², at least about 35 pmol/cm², at least about 40 pmol/cm², at least about 45 pmol/cm², at least about 50 pmol/cm², at least about 55 pmol/cm², at least about 60 pmol/cm², at least about 65 pmol/cm², at least about 70 pmol/cm², at least about 75 pmol/cm², at least about 80 pmol/cm², at least about 85 pmol/cm², at least about 90 pmol/cm², at least about 95 pmol/cm², at least about 100 pmol/cm², at least about 125 pmol/cm², at least about 150 pmol/cm², at least about 175 pmol/cm², at least about 200 pmol/cm², at least about 250 pmol/cm², at least about 300 pmol/cm², at least about 350 pmol/cm², at least about 400 pmol/cm², at least about 450 pmol/cm², at least about 500 pmol/cm², at least about 550 pmol/cm², at least about 600 pmol/cm², at least about 650 pmol/cm², at least about 700 pmol/cm², at least about 750 pmol/cm², at least about 800 pmol/cm², at least about 850 pmol/cm², at least about 900 pmol/cm², at least about 950 pmol/cm², at least about 1000 pmol/cm² or more.

Alternatively, the density of oligonucleotide on the external surface of the SNA is measured by the number of oligonucleotides on the external surface of a SNA. With respect to the surface density of oligonucleotides on the external surface of a SNA of the disclosure, it is contemplated that a SNA as described herein comprises about 1 to about 2,000 oligonucleotides on its external surface. In various embodiments, a SNA comprises about 10 to about 200, or about 10 to about 190, or about 10 to about 180, or about 10 to about 170, or about 10 to about 160, or about 10 to about 150, or about 10 to about 140, or about 10 to about 130, or about 10 to about 120, or about 10 to about 110, or about 10 to about 100, or 10 to about 90, or about 10 to about 80, or about 10 to about 70, or about 10 to about 60, or about 10 to about 50, or about 10 to about 40, or about 10 to about 30, about 10 to about 20, or about 50 to about 200, or about 50 to about 150, or about 50 to about 100, or about 50 to about 80 oligonucleotides on its surface. In some embodiments, a SNA comprises about 80 to about 140 oligonucleotides on its surface. In further embodiments, a SNA comprises at least about 5, 10, 20, 30, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 polynucleotides on its surface. In further embodiments, a SNA consists of 5, 10, 20, 30, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 polynucleotides on its surface. In some embodiments, a liposomal SNA (which may, in various embodiments, be about or less than about 150 nanometers in diameter or about or less than about 100 nanometers in diameter or about or less than about 80 nanometers in diameter or about or less than about 70 nanometers in diameter) comprises about 10 to about 2,000 oligonucleotides, or about 10 to about 1,000 oligonucleotides, or about 10 to about 100 oligonucleotides, or about 10 to about 80 oligonucleotides, or about 10 to about 40 oligonucleotides on its surface. In some embodiments, a SNA comprises or consists of about 75 oligonucleotides (e.g., TLR9 agonist oligonucleotides) on its surface. In various embodiments, the number of TLR3 agonists encapsulated in a nanoparticle core is about 1 to about 10, or about 1 to about 9, or about 1 to about 8, or about 1 to about 7, or about 1 to about 6, or about 1 to about 5, or about 1 to about 4, or about 1 to about 3, or about 2 to about 10, or about 3 to about 10, or about 3 to about 9, or about 3 to about 8, or about 3 to about 7, or about 3 to about 6, or about 3 to about 5. In further embodiments, and as described herein, one or more additional oligonucleotides is encapsulated in the nanoparticle core in addition to the TLR3 agonist(s).

### Compositions

The disclosure includes compositions that comprise one or a plurality of spherical nucleic acid (SNA) comprising: (a) a nanoparticle core; (b) one or more toll-like receptor 3 (TLR3) agonists encapsulated in the nanoparticle core; and (c) a shell of oligonucleotides attached to the external surface of the nanoparticle core, the shell of oligonucleotides comprising one or more toll-like receptor 9 (TLR9) agonist oligonucleotides. In some embodiments, the composition is an antigenic composition. In some embodiments, the composition further comprises a pharmaceutically acceptable carrier. The term “carrier” refers to a vehicle within which the SNA as described herein is administered to a mammalian subject. The term carrier encompasses diluents, excipients, adjuvants and combinations thereof. Pharmaceutically acceptable carriers are well known in the art (see, e.g., Remington's Pharmaceutical Sciences by Martin, 1975).

Exemplary “diluents” include sterile liquids such as sterile water, saline solutions, and buffers (e.g., phosphate, tris, borate, succinate, or histidine). Exemplary “excipients” are inert substances that may enhance vaccine stability and include but are not limited to polymers (e.g., polyethylene glycol), carbohydrates (e.g., starch, glucose, lactose, sucrose, or cellulose), and alcohols (e.g., glycerol, sorbitol, or xylitol). Adjuvants include vaccine delivery systems (e.g., emulsions, microparticles, immune stimulating complexes (ISCOMS), or liposomes) that target associated antigens to antigen presenting cells (APC); and immunostimulatory adjuvants.

### Antigens

The disclosure provides spherical nucleic acids (SNAs) comprising (a) a nanoparticle core; (b) one or more toll-like receptor 3 (TLR3) agonists encapsulated in the nanoparticle core; and (c) a shell of oligonucleotides attached to the external surface of the nanoparticle core, the shell of oligonucleotides comprising one or more toll-like receptor 9 (TLR9) agonist oligonucleotides. In some embodiments, a SNA of the disclosure comprises an antigen. In various embodiments, the antigen is a peptide, a protein, or a combination thereof. In further embodiments, the antigen is a cancer-related antigen or a viral antigen. In some embodiments, the viral antigen is a coronavirus (e.g., a SARS-CoV-2) antigen. In some embodiments, the cancer is breast cancer, peritoneum cancer, cervical cancer, colon cancer, rectal cancer, esophageal cancer, eye cancer, liver cancer, pancreatic cancer, larynx cancer, lung cancer, skin cancer, ovarian cancer, prostate cancer, stomach cancer, testicular cancer, thyroid cancer, brain cancer, or a combination thereof.

In some embodiments, the antigen is encapsulated in the nanoparticle core. In some embodiments, the antigen is attached to the surface of the nanoparticle core, the antigen is attached to an oligonucleotide in the shell of oligonucleotides, or both (see, e.g., U.S. Patent Application Publication No. 2020/0384104, incorporated herein by reference in its entirety). In further embodiments, an antigen is both (i) encapsulated in the nanoparticle core and (ii) attached to the surface of the nanoparticle core and/or attached to an oligonucleotide in the shell of oligonucleotides. In some embodiments, the antigen that is encapsulated in a nanoparticle core is a tumor cell lysate. In some embodiments, the antigen that is attached to the surface attached to the surface of the nanoparticle core, the antigen is attached to an oligonucleotide in the shell of oligonucleotides, or both, is a prostate cancer antigen.

### Methods of Inducing an Immune Response

The disclosure includes methods for eliciting an immune response in a subject in need thereof, comprising administering to the subject an effective amount of an antigenic composition comprising one or more of the SNA as described herein. Unless otherwise indicated, the antigenic composition is an immunogenic composition.

The immune response raised by the methods of the present disclosure generally includes an antibody response, preferably a neutralizing antibody response, antibody dependent cell-mediated cytotoxicity (ADCC), antibody cell-mediated phagocytosis (ADCP), complement dependent cytotoxicity (CDC), and T cell-mediated response such as CD4+, CD8+. The immune response generated by the SNA (optionally comprising an antigen) as disclosed herein generates an immune response that recognizes, and preferably ameliorates and/or neutralizes, a target (e.g., a virus, a cancer cell) as described herein. Methods for assessing antibody responses after administration of an antigenic composition (immunization or vaccination) are known in the art and/or described herein. In some embodiments, the immune response comprises a T cell-mediated response (e.g., peptide-specific response such as a proliferative response or a cytokine response). In preferred embodiments, the immune response comprises both a B cell and a T cell response. Antigenic compositions can be administered in a number of suitable ways, such as intramuscular injection, subcutaneous injection, intradermal administration and mucosal administration such as oral or intranasal. Additional modes of administration include but are not limited to intravenous, intraperitoneal, intranasal administration, intra-vaginal, intra-rectal, and oral administration. A combination of different routes of administration in the immunized subject, for example intramuscular and intranasal administration at the same time, is also contemplated by the disclosure.

Antigenic compositions may be used to treat both children and adults, including pregnant women. Thus a subject may be less than 1 year old, 1-5 years old, 5-15 years old, 15-55 years old, or at least 55 years old. Preferred subjects for receiving the vaccines are the elderly (e.g., >55 years old, >60 years old, preferably >65 years old), and the young (e.g., <6 years old, 1-5 years old, preferably less than 1 year old). Additional subjects for receiving the vaccines or compositions of the disclosure include naïve (versus previously infected) subjects, currently infected subjects, or immunocompromised subjects.

Administration can involve a single dose or a multiple dose schedule. Multiple doses may be used in a primary immunization schedule and/or in a booster immunization schedule. In a multiple dose schedule the various doses may be given by the same or different routes, e.g., a parenteral prime and mucosal boost, or a mucosal prime and parenteral boost. Administration of more than one dose (typically two doses) is particularly useful in immunologically naive subjects or subjects of a hyporesponsive population (e.g., diabetics, or subjects with chronic kidney disease (e.g., dialysis patients)). Multiple doses will typically be administered at least 1 week apart (e.g., about 2 weeks, about 3 weeks, about 4 weeks, about 6 weeks, about 8 weeks, about 10 weeks, about 12 weeks, or about 16 weeks). Preferably multiple doses are administered from one, two, three, four or five months apart. Antigenic compositions of the present disclosure may be administered to patients at substantially the same time as (e.g., during the same medical consultation or visit to a healthcare professional) other vaccines.

In general, the amount of SNA in each dose of the antigenic composition is selected as an amount effective to induce an immune response in the subject, without causing significant, adverse side effects in the subject. Preferably the immune response elicited includes: neutralizing antibody response; antibody dependent cell-mediated cytotoxicity (ADCC); antibody cell-mediated phagocytosis (ADCP); complement dependent cytotoxicity (CDC); T cell-mediated response such as CD4+, CD8+, or a protective antibody response. Protective in this context does not necessarily require that the subject is completely protected against, e.g., infection. A protective response is achieved when the subject is protected from developing symptoms of disease. As described above, the immune response generated by the SNA as disclosed herein generates an immune response that recognizes, and preferably ameliorates and/or neutralizes, a target (e.g., a virus, a cancer cell) as described herein.

In some aspects, the disclosure provides a method of up-regulating activity of toll-like receptor 3 (TLR3) and toll-like receptor 9 (TLR9), the method comprising contacting a cell having TLR3 and TLR9 with a spherical nucleic acid (SNA) of the disclosure. In some embodiments, the method is performed in vitro. In some embodiments, the method is performed in vivo.

### Methods of Gene Regulation

In some aspects of the disclosure, an oligonucleotide associated with a SNA of the disclosure inhibits the expression of a gene. Thus, in some embodiments, a SNA performs both an immunotherapeutic function and a gene inhibitory function. Accordingly, in some embodiments the shell of oligonucleotides that is attached to the external surface of the nanoparticle core comprises one or more TLR9 agonist oligonucleotides and one or more inhibitory oligonucleotides designed to inhibit target gene expression. In some embodiments, one or more inhibitory oligonucleotides designed to inhibit target gene expression is encapsulated in the nanoparticle core along with the one or more TLR3 agonists. In some embodiments, one or more inhibitory oligonucleotides designed to inhibit target gene expression is both attached to the external surface of the nanoparticle core and encapsulated in the nanoparticle core.

Methods for inhibiting gene product expression provided herein include those wherein expression of the target gene product is inhibited by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or 100% compared to gene product expression in the absence of a SNA. In other words, methods provided embrace those which results in essentially any degree of inhibition of expression of a target gene product.

The degree of inhibition is determined in vivo from a body fluid sample or from a biopsy sample or by imaging techniques well known in the art. Alternatively, the degree of inhibition is determined in a cell culture assay, generally as a predictable measure of a degree of inhibition that can be expected in vivo resulting from use of a specific type of SNA and a specific oligonucleotide.

In various aspects, the methods include use of an oligonucleotide which is 100% complementary to the target polynucleotide, i.e., a perfect match, while in other aspects, the oligonucleotide is at least (meaning greater than or equal to) about 95% complementary to the polynucleotide over the length of the oligonucleotide, at least about 90%, at least about 85%, at least about 80%, at least about 75%, at least about 70%, at least about 65%, at least about 60%, at least about 55%, at least about 50%, at least about 45%, at least about 40%, at least about 35%, at least about 30%, at least about 25%, at least about 20% complementary to the polynucleotide over the length of the oligonucleotide to the extent that the oligonucleotide is able to achieve the desired degree of inhibition of a target gene product.

The percent complementarity is determined over the length of the oligonucleotide. For example, given an antisense compound in which 18 of 20 nucleotides of the inhibitory oligonucleotide are complementary to a 20 nucleotide region in a target polynucleotide of 100 nucleotides total length, the oligonucleotide would be 90 percent complementary. In this example, the remaining noncomplementary nucleotides may be clustered or interspersed with complementary nucleobases and need not be contiguous to each other or to complementary nucleotides. Percent complementarity of an inhibitory oligonucleotide with a region of a target nucleic acid can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656).

The oligonucleotide utilized in such methods is either RNA or DNA. The RNA can be an inhibitory oligonucleotide, such as an inhibitory RNA (RNAi) that performs a regulatory function, and in various embodiments is selected from the group consisting of a small inhibitory RNA (siRNA), a single-stranded RNA (ssRNA), and a ribozyme. Alternatively, the RNA is microRNA that performs a regulatory function. The DNA is, in some embodiments, an antisense-DNA. In some embodiments, the RNA is a piwi-interacting RNA (piRNA).

### Uses of SNAs to Treat a Disorder

In some embodiments, a SNA of the disclosure is used to treat, attenuate, or prevent a disorder. Thus, in some aspects, the disclosure provides methods of treating or preventing a disorder comprising administering an effective amount of a SNA of the disclosure to a subject (e.g., a human subject) in need thereof, wherein the administering treats the disorder. In various embodiments, the disorder is cancer, an infectious disease, a pulmonary disease, a gastrointestinal disease, a hematologic disease, a viral disease, an inflammatory disease, an autoimmune disease, a neurodegenerative disease, an inherited disease, cardiovascular disease, or a combination thereof.

### Therapeutic Agents

In some embodiments, an SNA of the disclosure further comprise a therapeutic agent, or a plurality thereof. The therapeutic agent is, in various embodiments, simply associated with an oligonucleotide in the shell of oligonucleotides attached to the external surface of the nanoparticle core of the SNA, and/or the therapeutic agent is associated with the nanoparticle core of the SNA, and/or the therapeutic agent is encapsulated in the SNA. In some embodiments, the therapeutic agent is associated with the end of an oligonucleotide in the shell of oligonucleotides that is not attached to the nanoparticle core (e.g., if the oligonucleotide is attached to the nanoparticle core through its 3′ end, then the therapeutic agent is associated with the 5′ end of the oligonucleotide). Alternatively, in some embodiments, the therapeutic agent is associated with the end of an oligonucleotide in the shell of oligonucleotides that is attached to the nanoparticle core (e.g., if the oligonucleotide is attached to the nanoparticle core through its 3′ end, then the therapeutic agent is associated with the 3′ end of the oligonucleotide). In some embodiments, the therapeutic agent is covalently associated with an oligonucleotide in the shell of oligonucleotides that is attached to the external surface of the nanoparticle core of the SNA. In some embodiments, the therapeutic agent is covalently associated with a linker or spacer that is attached to the external surface of the nanoparticle core of the SNA. In some embodiments, the therapeutic agent is non-covalently associated with an oligonucleotide in the shell of oligonucleotides that is attached to the external surface of the nanoparticle core of the SNA. However, it is understood that the disclosure provides SNAs wherein one or more therapeutic agents are both covalently and non-covalently associated with oligonucleotides in the shell of oligonucleotides that is attached to the external surface of the nanoparticle core of the SNA. It will also be understood that non-covalent associations include hybridization, protein binding, and/or hydrophobic interactions. In some embodiments, a therapeutic agent is administered separately from a SNA of the disclosure. Thus, in some embodiments, a therapeutic agent is administered before, after, or concurrently with a SNA of the disclosure to treat a disorder.

Therapeutic agents contemplated by the disclosure include without limitation a protein (e.g., a therapeutic protein), a growth factor, a hormone, an interferon, an interleukin, an antibody or antibody fragment, a small molecule, a peptide, an antibiotic, an antifungal, an antiviral, a chemotherapeutic agent, or a combination thereof.

The term “small molecule,” as used herein, refers to a chemical compound or a drug, or any other low molecular weight organic compound, either natural or synthetic. By “low molecular weight” is meant compounds having a molecular weight of less than 1500 Daltons, typically between 100 and 700 Daltons.

### EXAMPLES

### Example 1

This Example describe the synthesis of SNAs that incorporate both a TLR3 agonist (polyinosinic:polycytidylic acid, poly(I:C)) and TLR9 agonist (CpG oligonucleotide) on the same liposomal scaffold. In this design, CpG comprises the SNA oligonucleotide shell, and poly(I:C) is encapsulated in the liposome core. These dual-TLR activating SNAs efficiently co-delivered high quantities of both agonists to the same target cell, yielding enhanced immunostimulation in various murine and human antigen-presenting cells (APCs). Moreover, co-delivery of TLR agonists using the SNA both synchronized and prolonged the duration of costimulatory molecule and major histocompatibility complex (MHC) expression in APCs, which has been shown to be important for efficient downstream immune responses. Taken together, these observations provide a strategy for potently activating immune cells and increasing the efficiency of their activation, which informs the design of nanomaterials for highly potent immunotherapies.

TLR3/9 SNAs were synthesized and their immunostimulatory effects were evaluated as compared to linear agonists or SNAs delivering single agents. Significantly, it was found that TLR3/9 SNAs induced more efficient immunostimulation over single agonist SNAs, by simultaneously broadening the window of expression and synchronizing the kinetics of MHC and costimulatory molecule presentation. Furthermore, these dual TLR-activating SNAs potently activated human immune cells, showing that the findings described herein are directly translatable to human disease. Together, this work provides a framework for designing nanoscale constructs that potently activate multiple TLR pathways concurrently and synergistically.

### Materials/Methods

Materials. DOPC lipid and CF-lipid were purchased from Avanti Polar Lipids as solutions in chloroform. TLR9 ligand ODN sequences, negative control sequences, and their dye-labeled modification sequences (Table 1) were synthesized on an ABI 3900 DNA oligo synthesizer with reagents purchased from Glen Research. TLR3 ligand poly(I:C) was purchased from InvivoGen. Cy3-labeled poly(I:C) was prepared using Label IT Nucleic Acid Labeling Kits (Mirus Bio) following the manufacturer's protocol. The cell-culture medium was prepared using RPMI 1640-GlutaMAX (Gibco) supplemented with heat-inactivated fetal bovine serum (10%, Atlanta Biologicals, processed by shaking at 100 rpm at 56° C. for 30 minutes on a temperature-controlled shaker), penicillin-streptomycin (100 U mL⁻¹, Gibco), and sodium pyruvate (1 mM, Gibco).

TABLE 1
Molecular weight distribution of as-received
poly(I:C) as calculated from GPC data.

| | | Poly(I:C) after extrusion |
|---|---|---|
| | Original Poly(I:C) | through 50 nm filters |
| M_{N} (g/mol) | 134,350 | 128,713 |
| M_{W} (g/mol) | 161,140 | 155,869 |
| M_{Z} (g/mol) | 199,809 | 197,819 |
| PDI (M_{W}/M_{N}) | 1.20 | 1.21 |

Cell lines. Mouse splenocytes were collected from male C57BL/6J wild-type mice purchased from The Jackson Laboratory. After spleens were collected, they were processed by smashing using a syringe plunger and washed with PBS through a cell strainer. After centrifuging at 500 g for 5 minutes, the supernatant was removed, and the cells were resuspended in ammonium-chloride-potassium lysing buffer (2 mL, Gibco) for 2 minutes. Cells were washed again with PBS and resuspended in the cell culture medium. Mouse bone marrow-derived DCs were processed from C57BL/6J wild-type mice following the literature protocol,⁵⁴ and cells were cultured in cell culture medium supplemented with recombinant mouse granulocyte-macrophage colony-stimulating factor (20 ng mL⁻¹, eBioScience). Fresh human normal PBMCs were purchased from ZenBio, and the cells were used within 12 hours after receiving.

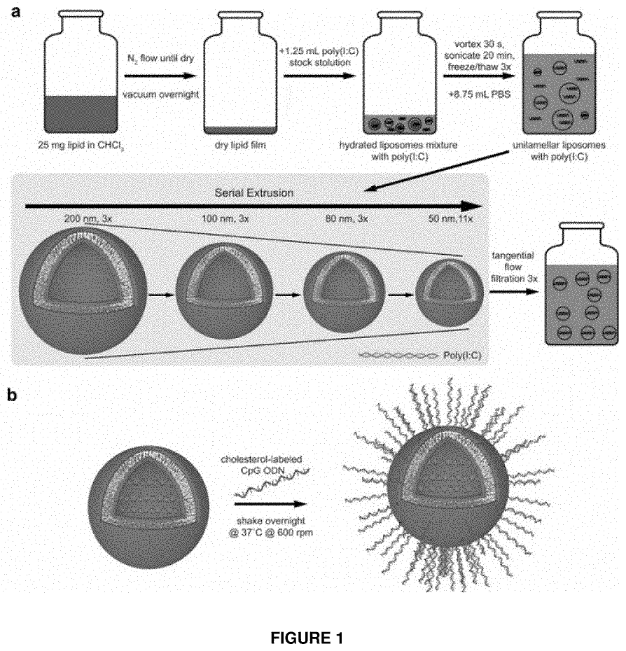

Preparation of liposomal SNAs. To make poly(I:C) encapsulated liposomes (FIG. 1a), a chloroform solution of DOPC lipid (1 mL, 25 mg mL⁻¹) was transferred into a glass vial, and the lipid was dried under nitrogen gas for 30 min and then under vacuum overnight to remove the organic solvent and generate a thin lipid film. The film was then rehydrated in poly(I:C) solution (1.25 mL, 20 mg mL⁻¹) prepared following the manufacturer's protocol. The suspension was vortexed for 30 seconds and sonicated for 20 minutes. After 3 freeze-thaw cycles, PBS (8.75 mL) was added, and the suspension underwent serial extrusion of 200-nm, 100-nm, 80-nm, and 50-nm track-etch membranes to obtain uniformly sized liposomes. Tangential flow filtration with MWCO 300 kDa fiber filter (Spectrum) was performed three times after the 200 nm and 50 nm extrusion steps, each with ten times the sample volume, to remove free poly(I:C). Empty liposomes were prepared via a similar procedure with PBS instead of a poly(I:C) solution. Poly(I:C) encapsulation was measured by UV absorption at 260 nm using UV/visible spectroscopy (Agilent) after incubating liposomes in 1% Triton-X solution for 1 hour to break apart the liposomes; the same treatment was performed with empty liposomes as a control. The final concentration of lipid was determined by a phosphatidylcholine assay (Sigma-Aldrich) following the manufacturer's protocol. To functionalize oligonucleotides onto the surface of liposomes, 3′-cholesterol-functionalized oligonucleotides (100 μM) were mixed with a predetermined amount of liposomes. Samples were held overnight at 37° C. under shaking at 500 rpm.

Materials characterization. Agarose gel electrophoresis was performed with 1% agarose (Sigma) in 1×TBE (ThermoFisher Scientific) under 120 V for 1 hour and was imaged using Typhoon Gel Imager (GE). Gel permeation chromatography (GPC, Agilent/Waytt) analysis was performed with poly(I:C) in PBS (100 uL, 1 mg mL-1). Dynamic light scattering (DLS) and zeta potential measurements were performed using a Malvern Zetasizer Nano with approximately 10 nM samples by particle. Cryo-TEM was performed with a JEOL 3200FS TEM equipped with an in-column energy filter (omega filter) and outfitted with a Gatan K2 Summitt Direct Electron Detector. Imaging was performed under 300 kV accelerating voltage. Cryo-TEM samples were prepared by loading the sample (4 μL, 100 μM CpG SNA stock solution or 1.33 μM liposome solution) onto 300-mesh gold TEM grids with lacey carbon (for liposomes and SNAs) or C-flat holey carbon (for poly(I:C)) films (Electron Microscopy Sciences) using an FEI Vitrobot Mark IV with its chamber equilibrated at 8° C. and 100% humidity. The samples were blotted for 5 seconds and plunged into liquid ethane before they were transferred and stored in liquid nitrogen.

Confocal microscopy and image analysis. Cells were fixed in paraformaldehyde solution (4% in PBS, diluted from a 32% aqueous solution from Electron Microscopy Sciences) for 15 minutes, and incubated in Hoechst 33342 (1 μg mL⁻¹, Invitrogen, FluoroPure grade) for nucleus staining. Confocal microscopy was performed using a Zeiss LSM800 with Airyscan module.⁵⁵ Data processing and colocalization calculations were performed using the Zeiss Zen Blue and an in-house Matlab script. A total of 25 cells were included in the analysis for each sample. The data processing procedure and Manders overlap coefficients^{56, 57} calculations were carried out as described in a previous publication.²⁰ Three-component overlap coefficients were calculated as:

$${r}_{3} = \frac{{\sum }_{i} ( {R}_{i} \cdot {G}_{i} \cdot {P}_{i} )}{{( {\sum }_{i} {( {R}_{i} )}^{3} \cdot {\sum }_{i} {( {G}_{i} )}^{3} \cdot {\sum }_{i} {( {P}_{i} )}^{3} )}^{\frac{1}{3}}}$$

where Rᵢ, Gᵢ, and Pᵢ are the grayscale values of the individual voxels i of the color components R (Cy5-CpG), G [Cy3-poly(I:C)], and P (CF-lipid).

Immunostimulation study. Mouse splenocytes and human PBMCs were used for the immune activation study. Cells (2×10⁶) were incubated with SNA or TLR agonist containing cell culture medium [200 μL, CpG (100 nM) and/or poly(I:C) (1.3 μg mL-1)] for 24 hours in 1.2 mL microtubes. The APC maturation kinetics study was performed following previous work.¹⁸ In brief, mouse bone marrow-derived DCs (1×10⁶) were incubated with SNA or TLR agonist containing cell culture medium [200 μL, CpG (2.5 μM) and/or poly(I:C) (16.3 μg mL⁻¹)] for 2 hours at 37° C. in 1.2 mL microtubes, then the cells were washed twice with fresh medium and incubated for up to 48 hours.

Flow cytometry. All antibodies and staining buffers were purchased from BD Biosciences. All antibodies were mixed and diluted to 1:200 in staining buffer. Cells were stained with antibody solutions for 15 minutes at 4° C. Mouse bone marrow-derived DCs were stained with antibody solution containing rat anti-mouse CD11 b, CD86, and I-A/I-E (MHCII), and hamster anti-mouse CD11c and CD80. Mouse splenocytes were stained with antibody solution containing rat anti-mouse CD11 b, CD19, CD45R/B220, CD86, F4/80, and MHCII, and hamster anti-mouse CD11c and CD80. Human PBMCs were stained with antibody solution containing anti-human Lin1 (CD3, CD14, CD16, CD19, CD20, CD56), mouse anti-human CD1c, CD11c, CD40, CD80, CD86, CD123, CD141, and HLA-DR. All cells were then fixed with 4% paraformaldehyde in PBS for 20 minutes at room temperature and stored at 4° C. for less than 24 hours prior to flow cytometry experiments.

Flow cytometry was performed with a BD LSR Fortessa flow cytometer system. A total of 30,000 singlet events were collected for each sample. Mouse bone marrow-derived DCs were gated with the CD11c⁺ population. Splenocyte APC populations were gated following previous work.¹⁶ In brief, pDCs were gated as B220⁺, CD11c^{med}, and CD19⁻; cDCs were gated as CD11c⁺, CD19⁻, and B220⁻; B cells were gated as CD19⁺, and B220⁺; and macrophages were gated as CD11b+, and F4/80+. For human PBMCs, the DC subpopulations were gated following the literature.⁵² In brief, BDCA-1 cDCs were gated as Lin1⁻, HLADR⁺, CD123⁻, CD11c⁺, and CD1c⁺; BDCA-2 pDCs were gated as Lin1⁻; HLA-DR⁺, and CD123⁺; and BDCA-3 cDCs were gated as Lin1⁻, HLADR⁺, CD123⁻, CD1c⁻, and CD141⁺. Color compensation was performed using AbC total antibody compensation bead kit (ThermoFisher Scientific). Data analysis was performed using FlowJo (BD).

Cytokine production analysis. Cytokine production was measured using Luminex 200, and the magnetic bead-based immunoassays were performed using ProcartaPlex kits purchased from ThermoFisher Scientific. The cell supernatants were collected from the same PBMC activation treatment experiments as used for the flow cytometry study. The cell culture supernatants were used with no further dilution. Sample processing and measurements were performed following the manufacturer's specific protocols. Data analysis was performed using Milliplex Analyst software.

Synthesis of oligonucleotides: Oligonucleotides were synthesized using an ABI 3900 DNA oligo synthesizer on a standard controlled pore glass (CPG) solid phase support following the manufacturer's protocol. All modified phosphoramidites were purchased from Glen Research. The products were purified by reverse phase high-performance liquid chromatography (HPLC). Syntheses were verified by matrix-assisted laser desorption/ionization-time-of-flight mass spectrometry (MALDI-TOF), and the concentrations were determined by UV-visible spectroscopy. A complete list of oligonucleotides synthesized can be found in Table 2 with their corresponding calculated and measured molecular weights.

TABLE 2
List of oligonucleotide sequences used herein.

| | | | Expected | Measured |
|---|---|---|---|---|
| | | SEQ | Mass | Mass |
| Strand | DNA sequence from 5′ to 3′ | ID NO: | (g/mol) | (g/mol) |
| ODN 1826-chol | tccatgacgttcctgacgtt-(Sp18)₂-chol | 1 | 7825 | 7850 |
| Linear ODN 1826 | tccatgacgttcctgacgtt | 2 | 6380 | 6364 |
| Cy3-CpG-B-chol | tccatgacgttcctgacgtt-(Sp18)-Cy3- | 3 | 8331 | 8359 |
| | (Sp18)-chol | | | |
| ODN 1826-Cy5-chol | tccatgacgttcctgacgtt-(Sp18)-Cy5- | 4 | 8357 | 8358 |
| | (Sp18)-chol | | | |
| Linear ODN 1826-Cy5 | tccatgacgttcctgacgtt-Cy5 | 5 | 7025 | 7025 |
| ODN 2138-chol | tccatgagcttcctgagctt-(Sp18)₂-chol | 6 | 7825 | 7824 |
| Linear ODN 2138 | tccatgagcttcctgagctt | 7 | 6380 | 6380 |
| ODN 7909-chol | tcgtcgttttgtcgttttgtcgtt-(Sp18)₂- | 8 | 9159 | 9183 |
| | chol | | | |
| Linear ODN 7909 | tcgtcgttttgtcgttttgtcgtt | 9 | 7698 | 7691 |
| ODN 2137-chol | tgctgcttttgtgcttttgtgctt-(Sp18)₂- | 10 | 9159 | 9185 |
| | chol | | | |
| Linear ODN 2137 | tgctgcttttgtgcttttgtgctt | 11 | 7698 | 7693 |

Notations and abbreviations:
1. All sequences have phosphorothioate backbones.
2. “chol” refers to 1-dimethoxytrityloxy-3-O-(N-cholesteryl-3-aminopropyl)-
triethyleneglycol-glyceryl-2-O-succinoyl-long chain alkylamino-CPG
(3′-Cholesteryl-TEG CPG).
3. “Sp18” refers to 18-O-dimethoxytritylhexaethyleneglycol, 1-[(2-cyanoethyl)-
(N,N-diisoproply)]-phosphoramidite (Spacer phosphoramidite 18).
4. “Cy3” refers to 1-[3-(4-monomethoxytrityloxy)propyl]-1′-[3-[(2-cyanoethyl)-
(N,N-diisopropyl) phosphoramidityl]propyl]-3,3,3′,3′-tetremethylindocarbocyanine
chloride (Cyanine 3 phosphoramidite).
5. “Cy5” refers to 1-[3-(4-monomethoxytrityloxy)propyl]-1′-[3-[(2-cyanoethyl)-
(N,N-diisopropyl) phosphoramidityl]propyl]-3,3,3′3′-tetramethylindodicarbocyanine
chloride (Cyanine 5 ohosphoramidite).

### Results and Discussion

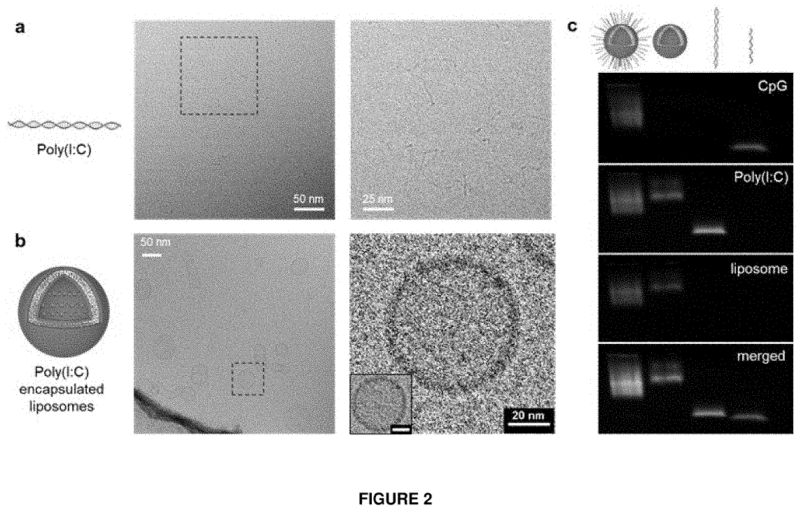

Characterization of poly(I:C) Prior to liposome synthesis, the poly(I:C) samples obtained from commercial sources were examined. For the as-received poly(I:C), gel permeation chromatography (GPC) returned a number average molecular weight (Mₙ) of approximately 134 kDa and weight average molecular weight (Mw) of approximately 161 kDa (polydispersity=M_{w}/Mₙ=1.20). Both agarose gel and GPC showed no significant size change of poly(I:C) before and after processing (i.e., sonication, three freeze-thaw cycles, and 50 nm extrusion, Table 1. Although transmission electron microscopy (TEM) only provides two-dimensional projections of potential three-dimensional structures, after qualitatively observing a large number of strands using cryo-TEM (FIG. 2a), none of the poly(I:C) observed had an end-to-end distance longer than 42 nm, suggesting that the end-to-end distance of the longest poly(I:C) would not be much longer than 42 nm, and thus these samples were suitable for encapsulation in liposomes with diameters of 50 nm.

Design and synthesis of dual-TLR activating SNAs. Liposome-based delivery systems are attractive for use in biological applications because their size is highly tailorable, and size is known to affect cellular uptake and biodistribution.^{36, 37} The commonly used liposome-based poly(I:C) formulation incorporates cationic lipids, either by adsorbing poly(I:C) on the surface of the liposomes³⁸ or by forming a layer-by-layer complex of poly(I:C) and the cationic lipids.^{39, 40} Although high loading of poly(I:C) can be achieved using this method, this approach is typically used to deliver nucleic acids to the cytosol rather than the endosomes. Moreover, because the cationic lipids interact with the negatively charged cellular membrane, this delivery method may lead to aggregation and the slow release of nucleic acids from the liposome while causing significant toxicity.⁴¹ Neutral liposomes, such as 1,2-dioleoyl-sn-glycero-3-phosphocholine (DOPC) liposomes, are generally non-toxic and have been previously used to encapsulate plasmid DNA by incorporating ethanol or calcium chloride in the solution to increase DNA loading. However, cellular delivery of nucleic acids with these constructs typically requires complete removal of those chemicals, which is challenging.⁴²⁻⁴⁴

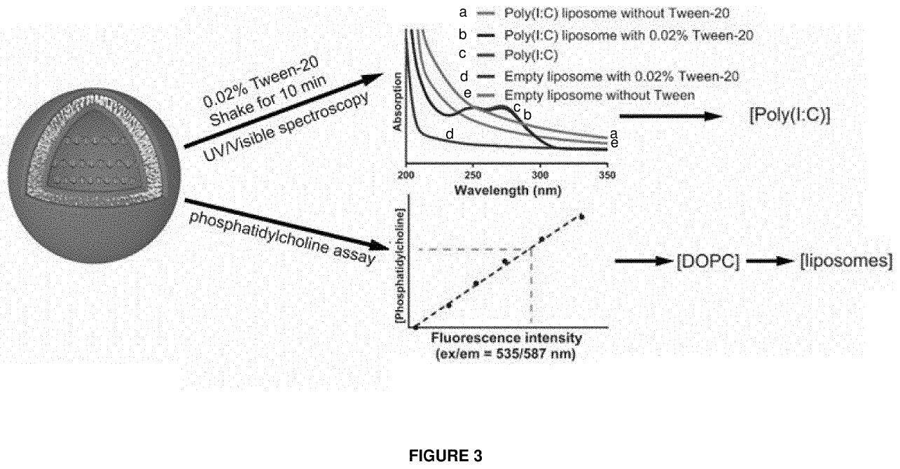

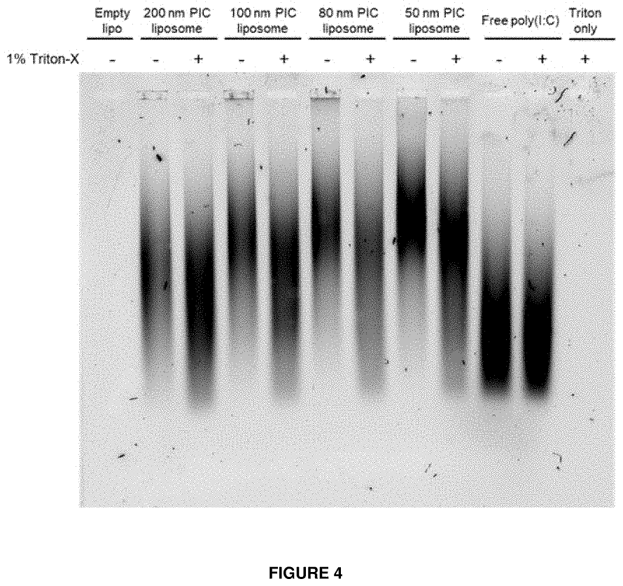

To circumvent these issues, a method for encapsulating multiple poly(I:C) strands in a single liposome without ethanol or calcium chloride was developed (FIG. 1). First, the commercial poly(I:C) was characterized and it was found to have a molecular weight of 134 kDa (Table 1) and a maximum length of 42 nm (FIG. 2a). This allowed it to fit into a 50-nm diameter liposome. Then, a relatively high concentration of poly(I:C) was used to rehydrate a relatively low concentration of lipid film to ensure high poly(I:C) encapsulation. This lipid suspension was then diluted 8 fold for ease of processing and to minimize material loss during extrusion. During the serial extrusion, samples were routinely collected and washed, and UV/Vis spectroscopy was used to determine how much poly(I:C) was encapsulated by disrupting the liposomes (FIG. 3). While the overall encapsulation efficiency and the number of poly(I:C) strands per particle decreases with decreasing liposome size, the local poly(I:C) concentration inside the liposomes was constant for the different size cores (Table 3). For a 50-nm diameter liposome, the poly(I:C) to liposome ratio was calculated to be approximately 3.15 strands per particle (22.83 μg dsRNA per pmol lipid), which is comparable to the reported ethanol- or calcium-based formulations.⁴⁴ Localization of poly(I:C) within the liposomes was confirmed by a combination of cryogenic transmission electron microscopy (Cryo-TEM) (FIG. 2b) and agarose gel electrophoresis (FIG. 2c and FIG. 4).

TABLE 3
Poly(I:C) loading in liposomes.

| | | Local | Local | Sample |
|---|---|---|---|---|
| Extrusion | | Concen- | Concen- | Concentration |
| size | Strands/ | tration | tration | (mg/mL, in 1.33 uM |
| (nm) | Particle | (μm) | (mg/mL) | 50-nm liposome) |
| 200 | 168.8 ± 8.4 | 66.9 | 10.8 | — |
| 100 | 16.4 ± 0.2 | 51.9 | 8.4 | — |
| 80 | 9.9 ± 0.3 | 61.6 | 9.9 | — |
| 50 | 3.2 ± 0.2 | 79.9 | 12.5 | 0.7 |

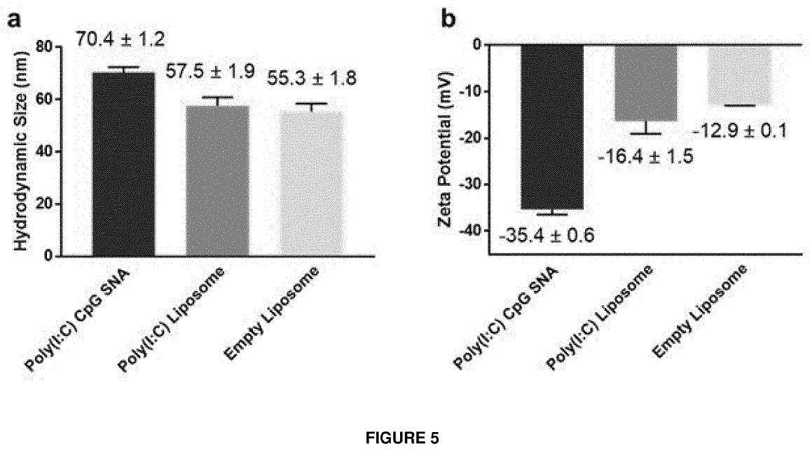

With these materials in hand, the poly(I:C)-encapsulated 50-nm liposomes were functionalized with cholesterol-labeled CpG ODN via membrane insertion to produce dual TLR-activating SNAs. An increase in hydrodynamic size and a decrease in zeta potential (FIG. 5) indicated successful ODN functionalization of the SNA surface. Additionally, the colocalization of the ODN shell, liposome core, and encapsulated poly(I:C) signals in an agarose gel (FIG. 2c) indicates that all three components are within a single nanoconstruct.

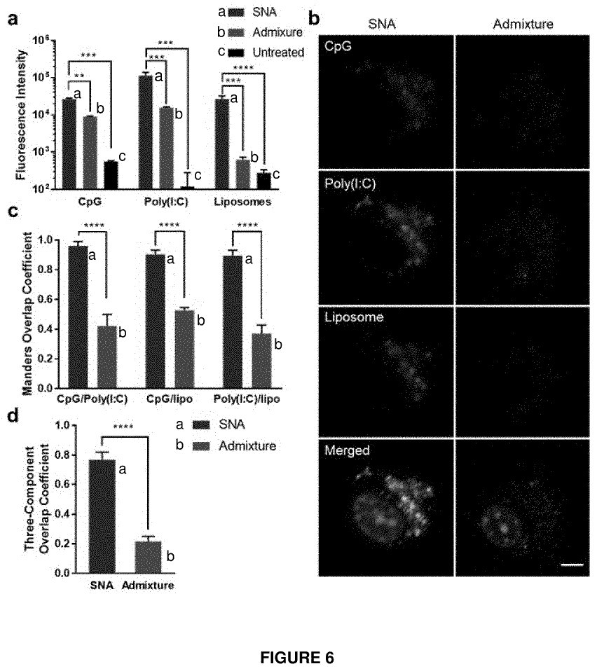

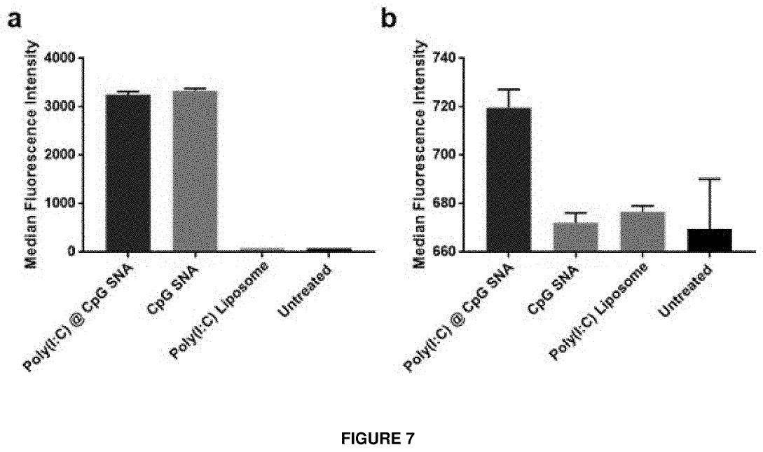

Co-delivery of Immunostimulatory Nucleic Acids via SNAs. The cellular uptake of the SNAs was studied using Cy5-labeled CpG ODN, Cy3-labeled poly(I:C), and DOPC liposomes containing 1% 1,2-dioleoyl-sn-glycero-3-phosphoethanolamine-N-carboxyfluorescein lipid (CF-lipid). After a 30 minutes incubation with dendritic cells (DCs), the cellular uptake of all three components was higher in the SNA format than for a fully soluble “admixture” of these components (FIG. 6a-b). Additionally, cells treated with SNAs both with and without poly(I:C) encapsulation showed similar cellular uptake of CpG ODN (FIG. 7a), indicating that uptake was primarily governed by the SNA architecture. In contrast, incubation with poly(I:C)-encapsulated SNAs showed significantly higher poly(I:C) uptake than with poly(I:C)-encapsulated liposomes (FIG. 7b), suggesting that preparing poly(I:C) in the SNA form significantly increased cellular uptake and facilitated delivery. The calculated Manders overlap coefficients for each pair of signals among the poly(I:C), CpG ODN, and liposome components were all over 0.9 for SNAs, which indicated very high colocalization (FIG. 6c). Conversely, the coefficients for admixtures were all below 0.6, indicating little colocalization. Additionally, the three-component overlap coefficient for SNAs was significantly higher (>3-fold) than that of the admixture, further confirming colocalization of all three components within cells (FIG. 6d). Taken together, these results showed that the SNA construct enhanced cellular uptake of poly(I:C) and CpG concomitantly, and therapeutic agents remained associated during cellular uptake.

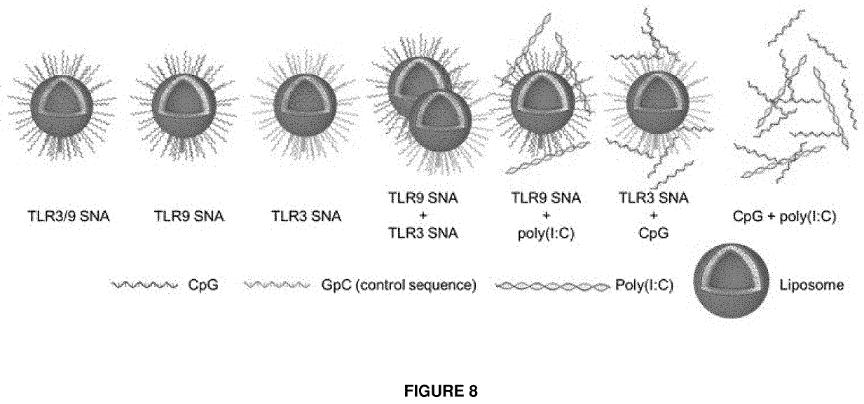

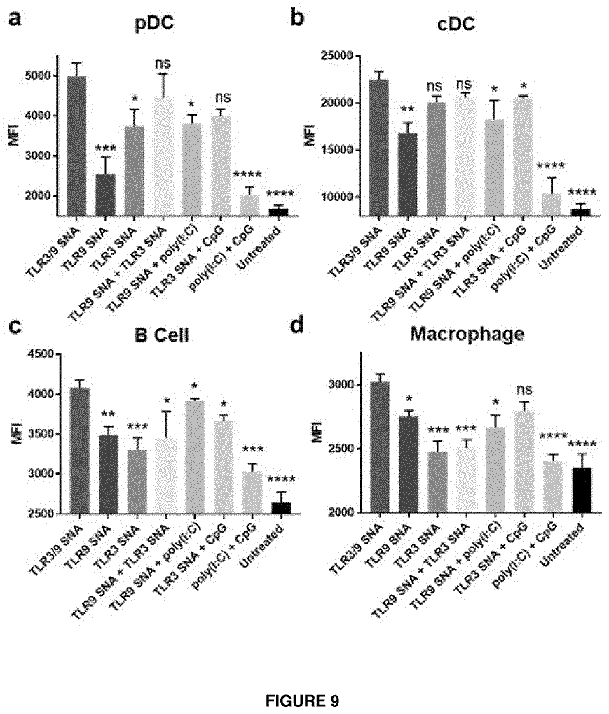

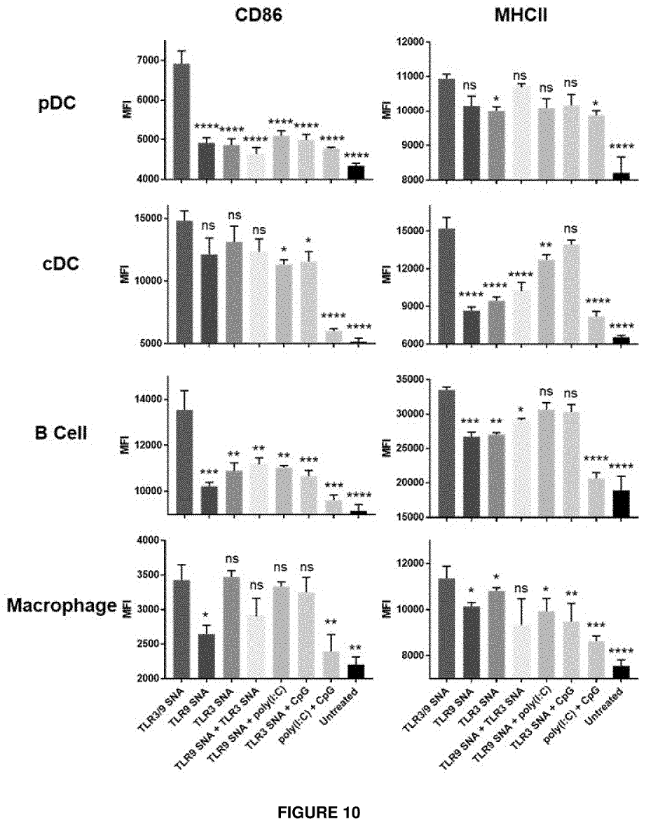

Activation of Murine Immune Cells. Downstream activation of the immune system requires the expression of molecules, such as CD80, CD86, and CD40 on the surface of mature APCs, which are required for T cell activation and survival. Importantly, TLR activation induces APC maturation and upregulation of these costimulatory molecules.^{45, 46} To assess the effect of SNAs on APC activation, mouse splenocytes were incubated with dual-TLR activating SNAs (TLR3/9 SNAs), single TLR activating SNAs (TLR3 SNAs or TLR9 SNAs), or mixtures of single TLR activating SNAs and linear TLR agonists (FIG. 8). Plasmacytoid dendritic cells (pDCs), conventional DC (cDCs), B cells, and macrophages were identified from the mouse splenocyte cells, and the APC costimulatory molecule expression levels were measured. Following a 24 hour incubation, cells treated with TLR3/9 SNAs showed significantly higher costimulatory molecule expression across all four APC subpopulations (FIG. 9 and FIG. 10). It was observed that pDCs and cDCs treated with TLR3 SNAs showed higher CD80 expression than those treated with TLR9 SNAs, while the opposite behavior was observed in B cells and macrophages. Different cell types may have different TLR expression levels, and treatment with dual-TLR agonists has the advantage of efficiently activating these different cells in a single treatment, which is important when these cells are closely associated in a complex mixture, as they are in most biological systems. Additionally, although mixing single-TLR activating SNAs with a second TLR agonist in its linear form boosted APC maturation, it was not to the same extent as observed in cells treated with dual-TLR activating TLR3/9 SNAs. Treatment with a mixture of TLR3 and TLR9 SNAs does not lead to high activation compared to the TLR3/9 SNAs. These suggest that co-delivery of the TLR agonists in the same SNA improves APC activation. Together, these results showed that TLR3/9 SNAs enhance the activation of a broader range of immune cells than single-TLR activating SNAs or admixtures of the components.

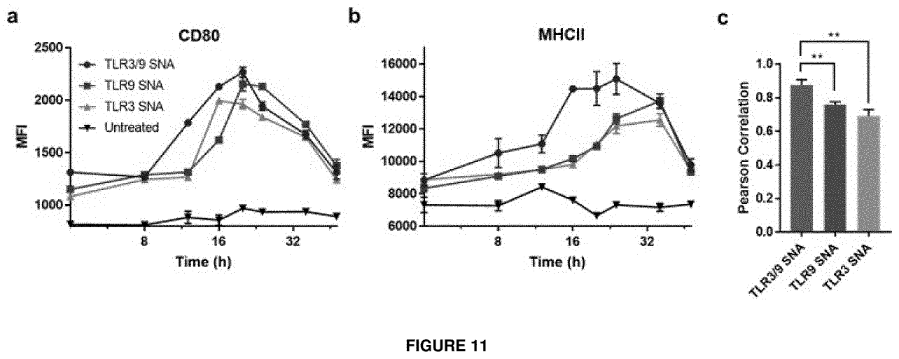

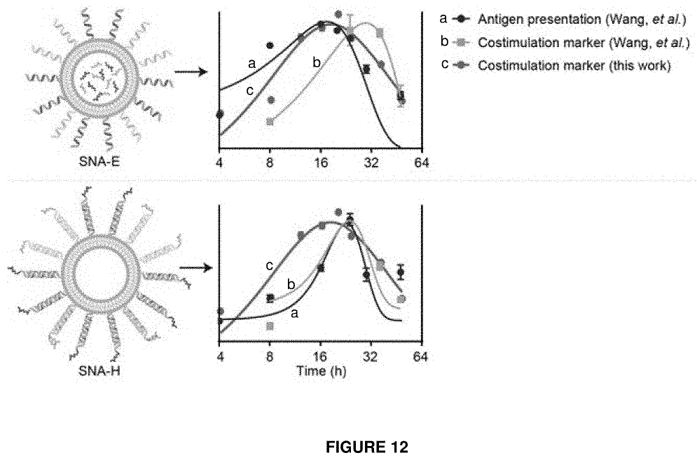

It has been shown that the expression levels of cytokine-related genes peak at different times after treatment with poly(I:C) or CpG;^{47, 48} however, the effect on costimulatory molecule expression kinetics has rarely been studied. Previously, it was found that costimulatory molecule expression kinetics are roughly the same after treating DCs with single-TLR activating CpG SNAs, regardless of SNA structure.¹⁸ It was hypothesized that introducing two TLR agonists would widen the window of costimulatory molecule expression, as we have previously found that this maximizes immune response.¹⁸ To test this, mouse bone marrow-derived DCs were incubated with SNAs containing CpG, poly(I:C), or both, and maturation marker expression levels were measured over time. It was found that cells treated with TLR3/9 SNAs began to express costimulatory molecules 12 hours after treatment, while cells treated with TLR9 SNAs or TLR3 SNAs did not show an increase in costimulatory expression until 16 hours after treatment (FIG. 11a). The decrease trends of the costimulatory molecule expression levels of these three treatments are similar. Additionally, TLR3/9 SNAs showed a broader time window for the expression of major histocompatibility complex (MHC) molecules (FIG. 11b), which can present antigen peptides for recognition by T cells. Expression of both MHC and costimulatory molecules is required for DCs to activate T cells and trigger downstream immune activation. Pearson correlation calculations revealed that cells incubated with TLR3/9 SNAs showed significantly higher synchronization of the expression of costimulatory molecules and MHC molecules (FIG. 11c). This suggested that treatment with TLR3/9 SNAs expanded the timeframe when both signals are present on these cells, which will lead to more effective T cell activation. Previously, it was reported that antigen presentation kinetics could be tuned with minimal effect on costimulation, by changing the antigen location on SNAs.¹⁸ Moreover, it has been found that the synchronization between antigen presentation and costimulation correlates with an enhanced immune response and tumor elimination in vivo.¹⁸ By comparing the activation kinetics of TLR3/9 SNAs to those of previous work (FIG. 12), it was found that the broadened window of costimulatory molecule expression determined herein completely covers the antigen presentation kinetics curves for all SNA structures tested previously. This suggested that TLR3/9 SNAs provide a framework for the development of antigen-containing materials with versatile and robust antigen presentation kinetics.

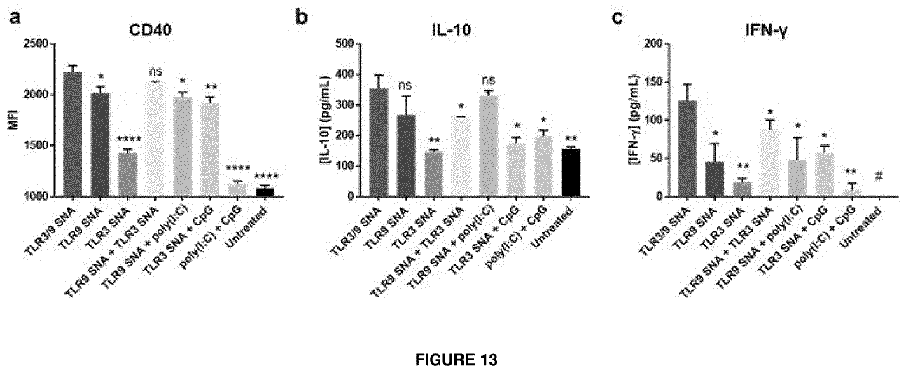

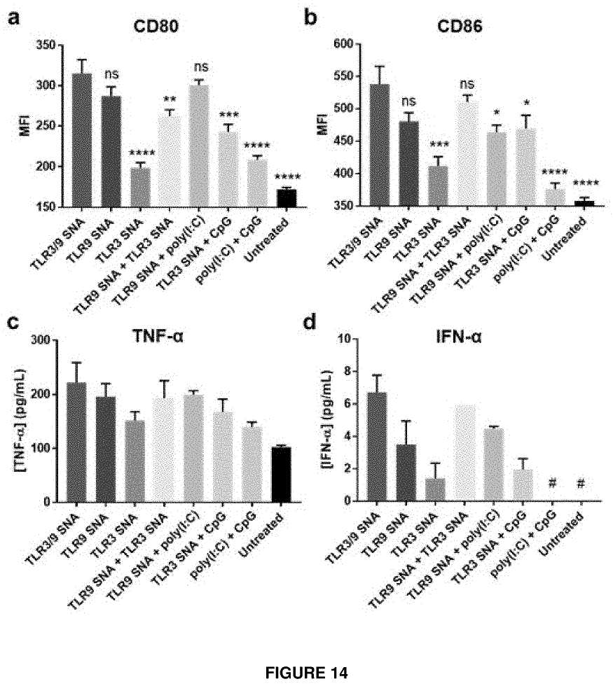

Activation of Human Immune Cells. While poly(I:C) has been shown to induce potent antitumor behaviors in many mouse models, translation to human cell lines is crucial since murine and human TLR3 may be activated differently.⁴⁹ In addition, TLR9 activation requires different CpG sequences for mice and humans.^{49, 50} To confirm that the enhanced immunostimulation behaviors induced by TLR3/9 SNAs in mice can be translated to humans, their ability to activate immune cells was tested using human peripheral blood mononuclear cells (PBMCs), which consists of various immune cells, including T cells, B cells, and DCs. After incubating PBMCs with SNAs for 24 hours, costimulatory molecule expression and cytokine secretions were measured (FIG. 13 and FIG. 14). Cells showed higher activation following treatment with TLR9 SNAs than TLR3 SNAs, and the addition of linear poly(I:C) to TLR9 SNAs did not significantly increase activation. However, cells treated with TLR3/9 SNAs showed a significant enhancement in most of the signals measured, indicating that these SNAs enhance immune activation in human cells.

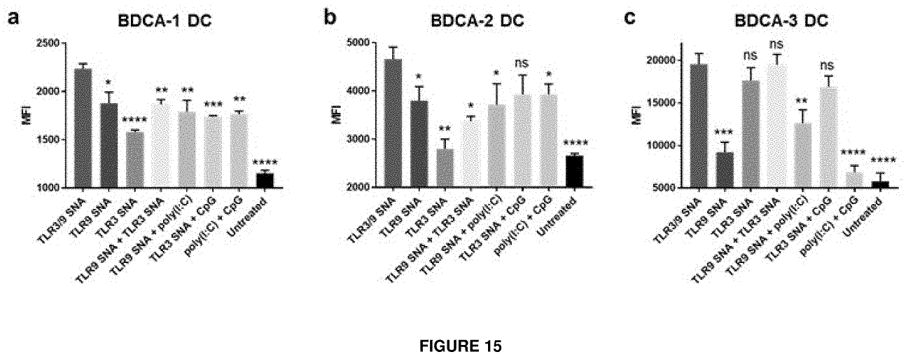

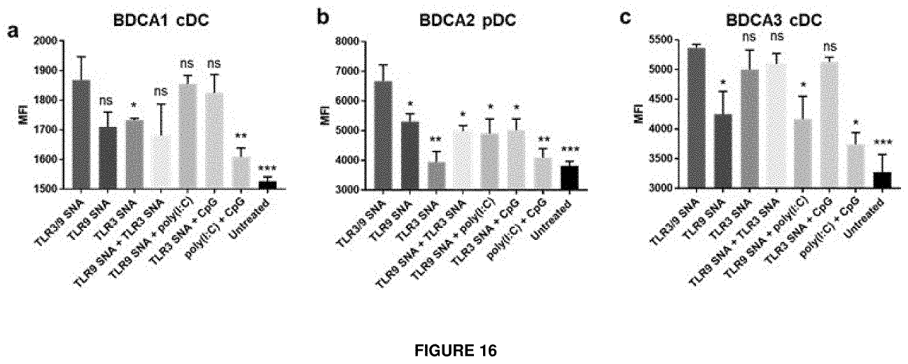

Furthermore, there are several different DC subpopulations in human PMBCs, which can be distinguished by their different blood DC antigen (BDCA) expressions.^{51, 52} These different DC subpopulations exhibit diverse TLR expression profiles,⁵¹ and respond to TLR agonists differently.⁵³ BDCA-1 DCs are conventional DCs (cDCs) that express a wide range of TLRs, BDCA-2 DCs are plasmacytoid DCs (pDCs) that express mainly TLR7 and TLR9, and BDCA-3 cDCs express high levels of TLR3.⁵² Using flow cytometry, we separated these DC subpopulations were separated and their costimulatory molecule expression levels were analyzed (FIG. 15 and FIG. 16). BDCA-2 DCs treated with TLR9 SNAs showed higher costimulatory molecule expression as compared to cells treated with TLR3 SNAs, while TLR3 SNAs activated BDCA-3 DCs better than TLR9 SNAs. These results agreed with the differences in TLR expression levels between each BDCA DC subtype, suggesting that PBMC activation by SNAs is TLR-specific. In addition, in all PBMC DC subpopulations, cells treated with TLR3/9 SNAs showed significantly enhanced costimulatory molecule expression as compared to single-TLR activating SNAs, their mixtures, or mixtures of single-TLR SNAs with a linear TLR agonist, similar to the behavior observed in murine APCs (FIG. 9). These results suggested that TLR3/9 dual-activating SNAs can be translated to human cells and enhance immune activation in both DC subpopulations and immune cells.

### CONCLUSIONS

The architecture of liposomal SNAs enabled efficient co-delivery of both TLR3 and TLR9 agonists. These dual-TLR activating SNAs significantly enhanced APC activation and increased cytokine secretions, in contrast to single TLR activating SNAs, their mixture, or their linear counterparts. This finding suggested that the co-delivery of the agonists as one entity induces the co-activation of the two TLRs in the same endosome, leading to improved synergistic immune activation. Moreover, dual-TLR activating SNAs simultaneously broadened the window of and synchronize the kinetics of costimulatory molecules and MHC molecules expressions, leading to prolonged effective immunostimulation, as compared to SNAs containing only one agonist. These behaviors indicated that dual-TLR activating SNAs can accommodate a broader range of antigens with different presentation kinetics, giving them the potential to act as potent cancer immunotherapeutics if tumor-associated antigens are incorporated into the scaffold. Similar synergistic immunostimulation behaviors by these TLR3/9 dually activating SNAs were observed in both murine and human immune cells. Therefore, these SNAs are translatable to target human diseases. Notably, the synthesis and characterization methods for encapsulating long nucleic acids into SNAs presented herein provide a framework for designing SNAs that deliver other types of long nucleic acids (e.g., plasmid DNAs and mRNAs) and thus expand the therapeutic applications that SNAs can target.

### REFERENCES

1. Akira, S.; Takeda, K., Toll-Like Receptor Signalling. Nat. Rev. Immunol. 2004, 4, 499-511.
2. Beutler, B., Inferences, Questions and Possibilities in Toll-Like Receptor Signalling. Nature 2004, 430, 257-263.
3. Rakoff-Nahoum, S.; Medzhitov, R., Toll-Like Receptors and Cancer. Nat. Rev. Cancer 2009, 9, 57-63.
4. Barbalat, R.; Ewald, S. E.; Mouchess, M. L.; Barton, G. M., Nucleic Acid Recognition by the Innate Immune System. Annu. Rev. Immunol. 2011, 29, 185-214.
5. Majer, O.; Liu, B.; Barton, G. M., Nucleic Acid-Sensing Tlrs: Trafficking and Regulation. Curr. Opin. Immunol. 2017, 44, 26-33.
6. Heil, F.; Hemmi, H.; Hochrein, H.; Ampenberger, F.; Kirschning, C.; Akira, S.; Lipford, G.; Wagner, H.; Bauer, S., Species-Specific Recognition of Single-Stranded RNA Via Toll-Like Receptor 7 and 8. Science 2004, 303, 1526-1529.
7. Krieg, A. M.; Yi, A.-K.; Matson, S.; Waldschmidt, T. J.; Bishop, G. A.; Teasdale, R.; Koretzky, G. A.; Klinman, D. M., CpG Motifs in Bacterial DNA Trigger Direct B-Cell Activation. Nature 1995, 374, 546-549.
8. Hemmi, H.; Takeuchi, O.; Kawai, T.; Kaisho, T.; Sato, S.; Sanjo, H.; Matsumoto, M.; Hoshino, K.; Wagner, H.; Takeda, K.; Akira, S., A Toll-Like Receptor Recognizes Bacterial DNA. Nature 2000, 408, 740-745.
9. Liu, L.; Botos, I.; Wang, Y.; Leonard, J. N.; Shiloach, J.; Segal, D. M.; Davies, D. R., Structural Basis of Toll-Like Receptor 3 Signaling with Double-Stranded RNA. Science 2008, 320, 379-81.
10. Leonard, J. N.; Ghirlando, R.; Askins, J.; Bell, J. K.; Margulies, D. H.; Davies, D. R.; Segal, D. M., The Tlr3 Signaling Complex Forms by Cooperative Receptor Dimerization. Proc. Natl. Acad. Sci. U.S.A 2008, 105, 258-63.
11. Schulz, 0.; Diebold, S. S.; Chen, M.; Naslund, T. I.; Nolte, M. A.; Alexopoulou, L.; Azuma, Y.-T.; Flavell, R. A.; Liljeström, P.; Reis e Sousa, C., Toll-Like Receptor 3 Promotes Cross-Priming to Virus-Infected Cells. Nature 2005, 433, 887-892.
12. Salem, M. L.; Kadima, A. N.; Cole, D. J.; Gillanders, W. E., Defining the Antigen-Specific T-Cell Response to Vaccination and Poly(I: C)/Tlr3 Signaling—Evidence of Enhanced Primary and Memory Cd8 T-Cell Responses and Antitumor Immunity. J. Immunother. 2005, 28, 220-228.
13. Alexopoulou, L.; Holt, A. C.; Medzhitov, R.; Flavell, R. A., Recognition of Double-Stranded RNA and Activation of NF-kB by Toll-Like Receptor 3. Nature 2001, 413, 732-738.
14. Mirkin, C. A.; Letsinger, R. L.; Mucic, R. C.; Storhoff, J. J., A DNA-Based Method for Rationally Assembling Nanoparticles into Macroscopic Materials. Nature 1996, 382, 607-609.
15. Cutler, J. I.; Auyeung, E.; Mirkin, C. A., Spherical Nucleic Acids. J. Am. Chem. Soc. 2012, 134, 1376-91.
16. Guan, C.; Chernyak, N.; Dominguez, D.; Cole, L.; Zhang, B.; Mirkin, C. A., RNA-Based Immunostimulatory Liposomal Spherical Nucleic Acids as Potent TLR7/8 Modulators. Small 2018, 14, e1803284.
17. Radovic-Moreno, A. F.; Chernyak, N.; Mader, C. C.; Nallagatla, S.; Kang, R. S.; Hao, L.; Walker, D. A.; Halo, T. L.; Merkel, T. J.; Rische, C. H.; Anantatmula, S.; Burkhart, M.; Mirkin, C. A.; Gryaznov, S. M., Immunomodulatory Spherical Nucleic Acids. Proc. Natl. Acad. Sci. U.S.A 2015, 112, 3892-7.
18. Wang, S.; Qin, L.; Yamankurt, G.; Skakuj, K.; Huang, Z.; Chen, P. C.; Dominguez, D.; Lee, A.; Zhang, B.; Mirkin, C. A., Rational Vaccinology with Spherical Nucleic Acids. Proc. Natl. Acad. Sci. U.S.A 2019, 116, 10473-10481.
19. Yamankurt, G.; Berns, E. J.; Xue, A.; Lee, A.; Bagheri, N.; Mrksich, M.; Mirkin, C. A., Exploration of the Nanomedicine-Design Space with High-Throughput Screening and Machine Learning. Nat. Biomed. Eng. 2019, 3, 318-327.
20. Huang, Z. N.; Cole, L. E.; Callmann, C. E.; Wang, S.; Mirkin, C. A., Sequence Multiplicity within Spherical Nucleic Acids. ACS Nano 2020, 14, 1084-1092.
21. Choi, C. H.; Hao, L.; Narayan, S. P.; Auyeung, E.; Mirkin, C. A., Mechanism for the Endocytosis of Spherical Nucleic Acid Nanoparticle Conjugates. Proc. Natl. Acad. Sci. U.S.A 2013, 110, 7625-30.
22. Prigodich, A. E.; Alhasan, A. H.; Mirkin, C. A., Selective Enhancement of Nucleases by Polyvalent DNA-Functionalized Gold Nanoparticles. J. Am. Chem. Soc. 2011, 133, 2120-2123.
23. Vance, D.; Martin, J.; Patke, S.; Kane, R. S., The Design of Polyvalent Scaffolds for Targeted Delivery. Adv. Drug Delivery Rev. 2009, 61, 931-939.
24. Zhu, Q.; Egelston, C.; Gagnon, S.; Sui, Y.; Belyakov, I. M.; Klinman, D. M.; Berzofsky, J. A., Using 3 TLR Ligands as a Combination Adjuvant Induces Qualitative Changes in T Cell Responses Needed for Antiviral Protection in Mice. J. Clin. Invest. 2010, 120, 607-616.
25. Zhu, Q.; Egelston, C.; Vivekanandhan, A.; Uematsu, S.; Akira, S.; Klinman, D. M.; Belyakov, I. M.; Berzofsky, J. A., Toll-Like Receptor Ligands Synergize through Distinct Dendritic Cell Pathways to Induce T Cell Responses: Implications for Vaccines. Proc. Natl. Acad. Sci. U.S.A 2008, 105, 16260-16265.
26. Wang, J.; Shao, Y.; Bennett, T. A.; Shankar, R. A.; Wightman, P. D.; Reddy, L. G., The Functional Effects of Physical Interactions among Toll-Like Receptors 7, 8, and 9. J. Biol. Chem. 2006, 281, 37427-37434.
27. Santiago-Raber, M.-L.; Dunand-Sauthier, I.; Wu, T.; Li, Q.-Z.; Uematsu, S.; Akira, S.; Reith, W.; Mohan, C.; Kotzin, B. L.; Izui, S., Critical Role of Tlr7 in the Acceleration of Systemic Lupus Erythematosus in Tlr9-Deficient Mice. J. Autoimmun. 2010, 34, 339-348.
28. Akbar, A. N.; Lord, J. M.; Salmon, M., Ifn-A and Ifn-B: A Link between Immune Memory and Chronic Inflammation. Immunol. Today 2000, 21, 337-342.
29. Miyazaki, M.; Kanto, T.; Inoue, M.; Itose, I.; Miyatake, H.; Sakakibara, M.; Yakushijin, T.; Kakita, N.; Hiramatsu, N.; Takehara, T.; Kasahara, A.; Hayashi, N., Impaired Cytokine Response in Myeloid Dendritic Cells in Chronic Hepatitis C Virus Infection Regardless of Enhanced Expression of Toll-Like Receptors and Retinoic Acid Inducible Gene-I. J. Med. Virol. 2008, 80, 980-988.
30. He, H.; Genovese, K. J.; Nisbet, D. J.; Kogut, M. H., Synergy of CpG Oligodeoxynucleotide and Double-Stranded RNA (Poly I:C) on Nitric Oxide Induction in Chicken Peripheral Blood Monocytes. Mol. Immunol. 2007, 44, 3234-3242.
31. Arsenault, R. J.; Kogut, M. H.; He, H., Combined CpG and Poly I:C Stimulation of Monocytes Results in Unique Signaling Activation Not Observed with the Individual Ligands. Cell. Signal. 2013, 25, 2246-2254.
32. Strandskog, G.; Skjæveland, I.; Ellingsen, T.; Jorgensen, J. B., Double-Stranded RNA- and CpG DNA-Induced Immune Responses in Atlantic Salmon: Comparison and Synergies. Vaccine 2008, 26, 4704-4715.
33. Barry, M. E.; Pinto-Gonzalez, D.; Orson, F. M.; McKenzie, G. J.; Petry, G. R.; Barry, M. A., Role of Endogenous Endonucleases and Tissue Site in Transfection and Cpg-Mediated Immune Activation after Naked DNA Injection. Hum. Gene. Ther. 1999, 10, 2461-80.
34. Bayyurt, B.; Tincer, G.; Almacioglu, K.; Alpdundar, E.; Gursel, M.; Gursel, I., Encapsulation of Two Different Tlr Ligands into Liposomes Confer Protective Immunity and Prevent Tumor Development. J. Control. Release 2017, 247, 134-144.
35. Liu, C.; Chu, X.; Yan, M.; Qi, J.; Liu, H.; Gao, F.; Gao, R.; Ma, G.; Ma, Y., Encapsulation of Poly I:C and the Natural Phosphodiester Cpg Odn Enhanced the Efficacy of a Hyaluronic Acid-Modified Cationic Lipid-Plga Hybrid Nanoparticle Vaccine in TC-1-Grafted Tumors. Int. J. Pharm. 2018, 553, 327-337.
36. Düzgüneş, N.; Nir, S., Mechanisms and Kinetics of Liposome-Cell Interactions. Adv. Drug Delivery Rev. 1999, 40, 3-18.
37. Bachmann, M. F.; Jennings, G. T., Vaccine Delivery: A Matter of Size, Geometry, Kinetics and Molecular Patterns. Nat. Rev. Immunol. 2010, 10, 787-796.
38. Zaks, K.; Jordan, M.; Guth, A.; Sellins, K.; Kedl, R.; Izzo, A.; Bosio, C.; Dow, S., Efficient Immunization and Cross-Priming by Vaccine Adjuvants Containing Tlr3 or Tlr9 Agonists Complexed to Cationic Liposomes. J. Immunol. 2006, 176, 7335-7345.
39. Nordly, P.; Rose, F.; Christensen, D.; Nielsen, H. M.; Andersen, P.; Agger, E. M.; Foged, C., Immunity by Formulation Design: Induction of High CD8+ T-Cell Responses by Poly(I:C) Incorporated into the Caf01 Adjuvant Via a Double Emulsion Method. J. Control. Release 2011, 150, 307-317.
40. Hafner, A. M.; Corthésy, B.; Merkle, H. P., Particulate Formulations for the Delivery of Poly(I:C) as Vaccine Adjuvant. Adv. Drug Delivery Rev. 2013, 65, 1386-1399.
41. Fillion, P.; Desjardins, A.; Sayasith, K.; Lagacé, J., Encapsulation of DNA in Negatively Charged Liposomes and Inhibition of Bacterial Gene Expression with Fluid Liposome-Encapsulated Antisense Oligonucleotides. Biochim. Biophys. Acta, Biomembr. 2001, 1515, 44-54.
42. Rivest, V.; Phivilay, A.; Julien, C.; Bélanger, S.; Tremblay, C.; Émond, V.; Calon, F., Novel Liposomal Formulation for Targeted Gene Delivery. Pharm. Res. 2007, 24, 981-990.
43. Bailey, A. L.; Sullivan, S. M., Efficient Encapsulation of DNA Plasmids in Small Neutral Liposomes Induced by Ethanol and Calcium. Biochim. Biophys. Acta, Biomembr. 2000, 1468, 239-252.
44. Jeffs, L. B.; Palmer, L. R.; Ambegia, E. G.; Giesbrecht, C.; Ewanick, S.; MacLachlan, I., A Scalable, Extrusion-Free Method for Efficient Liposomal Encapsulation of Plasmid DNA. Pharm. Res. 2005, 22, 362-372.
45. Lancaster, G. I.; Khan, Q.; Drysdale, P.; Wallace, F.; Jeukendrup, A. E.; Drayson, M. T.; Gleeson, M., The Physiological Regulation of Toll-Like Receptor Expression and Function in Humans. J. Physiol. 2005, 563, 945-55.
46. June, C. H.; Bluestone, J. A.; Nadler, L. M.; Thompson, C. B., The B7 and Cd28 Receptor Families. Immunol. Today 1994, 15, 321-31.
47. Luo, M.; Wang, H.; Wang, Z.; Cai, H.; Lu, Z.; Li, Y.; Du, M.; Huang, G.; Wang, C.; Chen, X.; Porembka, M. R.; Lea, J.; Frankel, A. E.; Fu, Y.-X.; Chen, Z. J.; Gao, J., A Sting-Activating Nanovaccine for Cancer Immunotherapy. Nat. Nanotechnol. 2017, 12, 648-654.
48. Li, Z. J.; Choi, D.-K.; Sohn, K.-C.; Lim, S. K.; Im, M.; Lee, Y.; Seo, Y.-J.; Kim, C. D.; Lee, J.-H., Induction of Interleukin-22 (11-22) Production in Cd4+ T Cells by II-17a Secreted from Cpg-Stimulated Keratinocytes. Ann. Dermatol. 2016, 28, 579-585.
49. Rehli, M., Of Mice and Men: Species Variations of Toll-Like Receptor Expression. Trends Immunol. 2002, 23, 375-8.
50. Heinz, S.; Haehnel, V.; Karaghiosoff, M.; Schwarzfischer, L.; Muller, M.; Krause, S. W.; Rehli, M., Species-Specific Regulation of Toll-Like Receptor 3 Genes in Men and Mice. J. Biol. Chem. 2003, 278, 21502-9.
51. Collin, M.; Bigley, V., Human Dendritic Cell Subsets: An Update. Immunology 2018, 154, 3-20.
52. Colletti, N. J.; Liu, H.; Gower, A. C.; Alekseyev, Y. O.; Arendt, C. W.; Shaw, M. H., Tlr3 Signaling Promotes the Induction of Unique Human BDCA-3 Dendritic Cell Populations. Front. Immunol. 2016, 7, 88.
53. Kadowaki, N.; Ho, S.; Antonenko, S.; Malefyt, R. W.; Kastelein, R. A.; Bazan, F.; Liu, Y. J., Subsets of Human Dendritic Cell Precursors Express Different Toll-Like Receptors and Respond to Different Microbial Antigens. J. Exp. Med. 2001, 194, 863-9.
54. Madaan, A.; Verma, R.; Singh, A. T.; Jain, S. K.; Jaggi, M., A Stepwise Procedure for Isolation of Murine Bone Marrow and Generation of Dendritic Cells. J. Biol. Methods 2014, 1, e1.
55. Huff, J., The Airyscan Detector from Zeiss: Confocal Imaging with Improved Signal-to-Noise Ratio and Super-Resolution. Nat. Methods 2015, 12, 1205.
56. Manders, E. M.; Stap, J.; Brakenhoff, G. J.; van Driel, R.; Aten, J. A., Dynamics of Three-Dimensional Replication Patterns During the S-Phase, Analysed by Double Labelling of DNA and Confocal Microscopy. J. Cell Sci. 1992, 103, 857-62.
57. Manders, E. M. M.; Verbeek, F. J.; Aten, J. A., Measurement of Co-Localization of Objects in Dual-Colour Confocal Images. J. Microsc. 1993, 169, 375-382.
58. Ferrer, J. R.; Wertheim, J. A.; Mirkin, C. A., Dual Toll-Like Receptor Targeting Liposomal Spherical Nucleic Acids. Bioconjug. Chem. 2019, 30 (3), 944.

1. A spherical nucleic acid (SNA) comprising:
(a) a nanoparticle core;
(b) one or more toll-like receptor 3 (TLR3) agonists encapsulated in the nanoparticle core; and
(c) a shell of oligonucleotides attached to the external surface of the nanoparticle core, the shell of oligonucleotides comprising one or more toll-like receptor 9 (TLR9) agonist oligonucleotides.

2. (canceled)

3. The SNA of claim 1, wherein the TLR3 agonist is polyinosinic:polycytidylic acid (poly(I:C)), polyadenylic:polyuridylic acid (poly(A:U)), viral double-stranded RNA, or a combination thereof.

4. (canceled)

5. (canceled)

6. (canceled)

7. The SNA of claim 1, wherein the SNA further comprises an antigen, wherein the antigen is a peptide, a protein, or a combination thereof.

8. (canceled)

9. The SNA of claim 7, wherein the antigen is a cancer-related antigen or a viral antigen.

10. The SNA of claim 7, wherein the antigen is encapsulated in the nanoparticle core.

11. The SNA of claim 7, wherein the antigen is a tumor cell lysate.

12. The SNA of claim 7, wherein the antigen is attached to the surface of the nanoparticle core, the antigen is attached to an oligonucleotide in the shell of oligonucleotides, or both.

13. The SNA of claim 12, wherein the antigen is a prostate cancer antigen.

14. The SNA of claim 1, wherein the nanoparticle core is a liposomal core.

15. (canceled)

16. (canceled)

17. (canceled)

18. The SNA of claim 1, wherein at least one TLR9 agonist oligonucleotide in the shell of oligonucleotides is attached to the external surface of the nanoparticle core through a lipid anchor group.

19. (canceled)

20. (canceled)

21. (canceled)

22. (canceled)

23. (canceled)

24. (canceled)

25. The SNA of claim 1, wherein the shell of oligonucleotides comprises one or more additional oligonucleotides.

26. (canceled)

27. (canceled)

28. The SNA of claim 1, further comprising one or more additional oligonucleotides encapsulated in the nanoparticle core.

29. The SNA of claim 28, wherein the one or more additional oligonucleotides encapsulated in the nanoparticle core comprises mRNA, plasmid DNA, a therapeutic oligonucleotide, a detection oligonucleotide, or a combination thereof.

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. An antigenic composition comprising the spherical nucleic acid (SNA) of claim 1, in a pharmaceutically acceptable carrier, diluent, stabilizer, preservative, or adjuvant, wherein the antigenic composition is capable of generating an immune response including antibody generation or a protective immune response in a mammalian subject.

43. (canceled)

44. A method of producing an immune response to cancer in a subject, comprising administering to the subject an effective amount of the antigenic composition of claim 42, thereby producing an immune response to cancer in the subject.

45. (canceled)

46. A method of treating a disorder comprising administering an effective amount of the spherical nucleic acid (SNA) of claim 1, to a subject in need thereof, wherein the administering treats the disorder.

47. The method of claim 46, wherein the disorder is cancer, an infectious disease, an autoimmune disease, or a combination thereof.

48. A method of up-regulating activity of toll-like receptor 3 (TLR3) and toll-like receptor 9 (TLR9) comprising contacting a cell having TLR3 and TLR9 with a spherical nucleic acid of claim 1.

49. (canceled)

50. (canceled)

51. (canceled)
